(19) United States
(12) Patent Application Publication
(10) Pub. No.: US 2024/0096476 A1
(43) Pub. Date: Mar. 21, 2024

(54) METHOD, THE COMPUTING DEIVCE, AND THE NON-TRANSITORY COMPUTER-READABLE RECORDING MEDIUM FOR PROVIDING COGNITIVE TRAINING

(71) Applicant: TAIPEI MEDICAL UNIVERSITY, Taipei City (TW)

(72) Inventor: CHAO-WEI CHEN, Taipei City (TW)

(21) Appl. No.: 18/467,722

(22) Filed: Sep. 14, 2023

Related U.S. Application Data
(60) Provisional application No. 63/375,849, filed on Sep. 15, 2022.
(60) Provisional application No. 63/514,567, filed on Jul. 19, 2023.

(51) Int. Cl. G16H 20/70; G06T 7/00

(52) U.S. Cl. CPC G16H 20/70; G06T 7/0012; G06T 2207/10088; G06T 2207/20081

(57) ABSTRACT

The present application provides a method for providing cognitive training by using a computing device. The method includes prompting a first target object for recognizing by a user; receiving a feedback response from the user by the computing device; determining a correctness of the feedback response by comparing the feedback response with a stored answer by the computing device; and adjusting a level or a type of a second target object based on a guideline associated with the correctness of the feedback response. The second target object is provided for recognizing by the user after the first target object is provided. In addition, the present application also provides a computing device for providing cognitive training and a non-transitory computer-readable recording medium capable of providing cognitive training.

### CROSS-REFERENCE TO RELATED APPLICATION

This non-provisional application claims priority under 35 U.S.C. § 119(e) on U.S. provisional Patent Application No(s). 63/375,849 filed on Sep. 15, 2022 and No(s). 63/514,567 filed on Jul. 19, 2023, the entire contents of which are hereby incorporated by reference.

### BACKGROUND OF THE INVENTION

### 1. Field of the Invention

The present disclosure relates to a method, a computing device, and a non-transitory computer-readable recording medium for providing training, and in particular to a method, a computing device, and a non-transitory computer-readable recording medium for providing cognitive training.

### 2. Description of the Related Art

Each patient with cognitive impairment would have a unique combination of dysfunctions in one or more cognitive domains, and the availability of a cognitive assistance system would be beneficial to patients.

Cognitive impairment is associated with a severe decline in brain functions with an impact on at least one domain of cognition, and it is commonly attributed to traumatic brain injury, degenerative processes, and other neurological pathology and environmental factors. The disorders associated with cognitive deficits such as Alzheimer's disease, vascular dementia, frontotemporal lobar degeneration, and dementia with Lewy bodies often result in behavioral and psychiatric issues.

Executive function is a multifaceted neuropsychological construct that can be defined as forming, maintaining, and shifting mental sets. It includes basic cognitive processes such as attentional control, cognitive inhibition, inhibitory control, working memory, and cognitive flexibility. The executive function enables people to successfully formulate goals, plan how to achieve them, and carry out the plans effectively which is essential for functional independence.

Patients suffer from executive function difficulties due to cognitive impairment and their quality of life has been drastically decreased.

### BRIEF SUMMARY OF THE INVENTION

The present disclosure provides a system and methods for cognitive rehabilitation of executive functions. The systems and methods for cognitive rehabilitation of executive functions could acquire environmental data, analyzes the data, and provides cognitive assistance based on the data.

To achieve at least the above feature, the present disclosure provides a method for providing cognitive training by using a computing device. The method includes prompting a first target object for recognizing by a user; receiving a feedback response from the user by the computing device; determining a correctness of the feedback response by comparing the feedback response with a stored answer by the computing device; and adjusting a level or a type of a second target object based on a guideline associated with the correctness of the feedback response. The second target object is provided for recognizing by the user after the first target object is provided.

In an embodiment, the first target object comprises at least one of a target image, a target text, and a target voice.

In an embodiment, the correctness of the feedback response comprises at least one of a match result that the feedback response exactly matches with the stored answer or not and a match ratio that the feedback response is close to the stored answer.

In an embodiment, the guideline is that the level of the second target object is upgraded when the match result is that the feedback response exactly matches with the stored answer or when the match ratio is greater than or equal to a threshold, and the level of the second target object is downgraded when the match result is that the feedback response does not exactly match with the stored answer or when the match ratio is less than the threshold.

In an embodiment, the guideline is that the type of the second target object which is more difficult than the first target object is determined when the match result is that the feedback response exactly matches with the stored answer or when the match ratio is greater than or equal to a threshold, and the type of the second target object which is easier than the first target object is determined when the match result is that the feedback response does not exactly match with the stored answer or when the match ratio is less than the threshold.

In an embodiment, the stored answer comprises at least one of a primary answer and a secondary answer.

In an embodiment, the step of prompting the first target object for recognizing by the user is performed by the computing device.

In an embodiment, the method for providing cognitive training further includes receiving a magnetic resonance imaging (MRI) of the user; inputting the magnetic resonance imaging into an image analysis model, and then an image analysis result corresponding to the magnetic resonance imaging being generated in real time by the image analysis model; and automatically determining the first target object based on the image analysis result by the computing device.

Furthermore, the present disclosure also provides a computing device for providing cognitive training. The computing device includes a signal receiving module, a processing module, a storage module, and a displaying module. The processing module is configured to couple with the signal receiving module, the storage module, and the displaying module. A code is stored in the storage module. After the processing module executes the code stored in the storage module, the computing device is able to execute steps such that any one of the methods for providing cognitive training as described above is carried out.

Furthermore, the present disclosure also provides a non-transitory computer-readable recording medium capable of providing cognitive training. After a computing device loading and executing a code stored in the non-transitory computer-readable recording medium, the non-transitory computer-readable recording medium is able to complete steps such that any one of the methods for providing cognitive training as described above is carried out.

### BRIEF DESCRIPTION OF THE DRAWINGS

FIG. 1A illustrates an exemplary design of the cognitive assistance system.

FIG. 1B is a flowchart of a method to convert data for cognitive compensation purpose.

FIG. 2 is an example of the system shown in FIG. 1A.

FIG. 3 is an example of the data analysis module and the data cognitive feedback module shown in FIG. 1A.

FIG. 4 is an exemplary flowchart of the system shown in FIG. 1A.

FIG. 5A illustrates an exemplary output screen generated by the cognitive assistance system (100) to provide cognitive assistance for executive function.

FIG. 5B shows an example of the cognitive feedback module.

FIG. 6A illustrates an exemplary output screen generated by the cognitive assistance system to provide cognitive assistance for executive function.

FIG. 6B illustrates an exemplary output screen generated by the system to provide cognitive assistance by converting facial image data into text data.

FIG. 6C depicts a functional magnetic resonance imaging of a patient with facial recognition impairment doing a face recognition task without device assistance.

FIG. 6D depicts a functional magnetic resonance imaging of a patient with facial recognition impairment doing a face recognition task device assistance.

FIG. 7 illustrates an exemplary output screen generated by the cognitive assistance system (100) to provide cognitive assistance related to language use.

FIG. 8 illustrates an exemplary output screen generated by the cognitive assistance system (100) to provide cognitive assistance related to navigation.

FIG. 9 illustrates an exemplary schedule setting screen of the cognitive assistance system.

FIG. 10 is a flowchart of a method to convert one or more data type into one or more data type for cognitive compensation purpose.

FIG. 11 illustrates an exemplary output screen generated by the cognitive assistance system to provide cognitive assistance.

FIG. 12 is a flowchart of a method related to execution of cognitive intervention.

FIG. 13 is a flowchart of a method related to cognitive monitoring.

FIG. 14 is a flowchart of an exemplary embodiment of method related to cognitive monitoring.

FIG. 15 illustrates an exemplary output screen generated by the cognitive assistance system (100) for cognitive monitoring.

FIG. 16 depicts a flowchart showing the cognitive functioning of a patient with facial recognition impairment doing a face recognition task without device assistance.

FIG. 17 illustrates an exemplary output screen generated by the cognitive assistance system for determination of compensation strategy and/or intervention strategy.

FIG. 18 is a flowchart of a method related to update of algorithm for the cognitive assistance system.

FIG. 19 is a flowchart of a method to convert data for cognitive compensation purpose and for execution of cognitive intervention.

FIG. 20 depicts a computer system that could serve as the system for the cognitive assistance system to be operated on.

FIG. 21 depicts a wearable cognitive prosthesis system that could serve as a cognitive assistance system.

FIG. 22 that provides customized cognitive assistance based on the patient's specific cognitive domain impairment depicts a wearable cognitive prosthesis system that, in some embodiments, could serve as a cognitive assistance system.

FIG. 23 depicts an embodiment of the cognitive prosthesis system.

FIG. 24 depicts an embodiment of the cognitive prosthesis system.

FIG. 25 illustrates an embodiment of a smart cognitive assistant.

FIG. 26 shows exemplary output screens of the smart cognitive assistant.

FIG. 27A illustrates the system that further includes a calibration module.

FIG. 27B shows an exemplary screen output of the calibration module.

FIG. 28 shows an example of a shopping module including a set of rehab tools, which allows a user to perform more complicated rehab tasks.

FIG. 29 shows the steps of the process of the shopping module.

FIG. 30 illustrates a computing device for providing cognitive training.

FIG. 31 illustrates a method for providing cognitive training.

FIG. 32 illustrates another method for providing cognitive training.

### DETAILED DESCRIPTION OF THE INVENTION

The present disclosure is further described in the following detailed description of exemplary embodiments, together with the accompanying figures. The same reference numbers in different drawings may identify the same or similar elements. Besides, the present disclosure may, however, be embodied in many different forms and should not be construed as being limited to the embodiments set forth herein. Rather, these embodiments are provided so that the present disclosure will be thorough and complete, and will fully convey the scope of the invention to those skilled in the art. In the figures, the shapes and dimensions of elements may be exaggerated for clarity, and the same reference numerals will be used throughout to designate the same or like components.

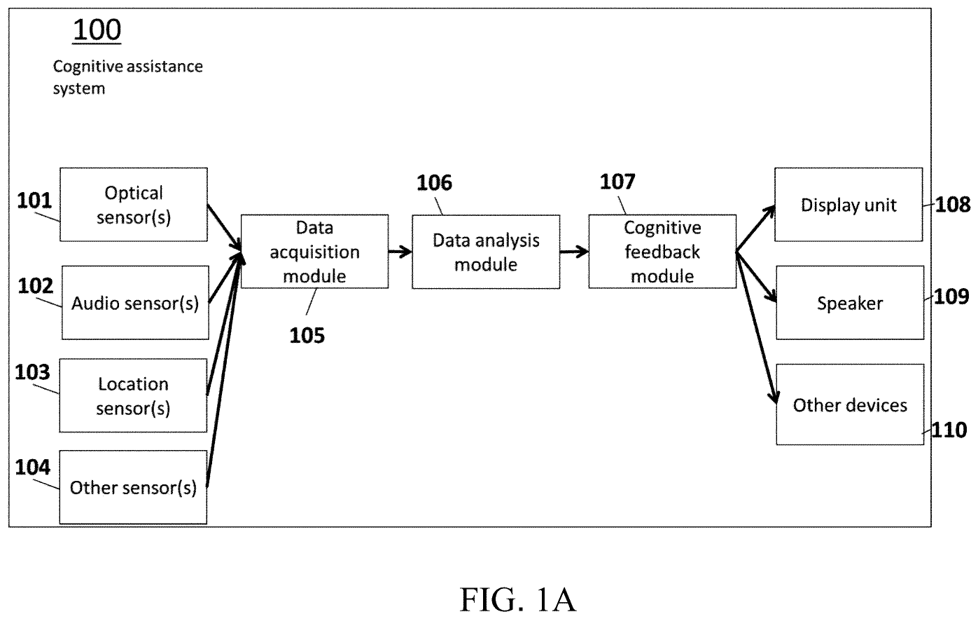

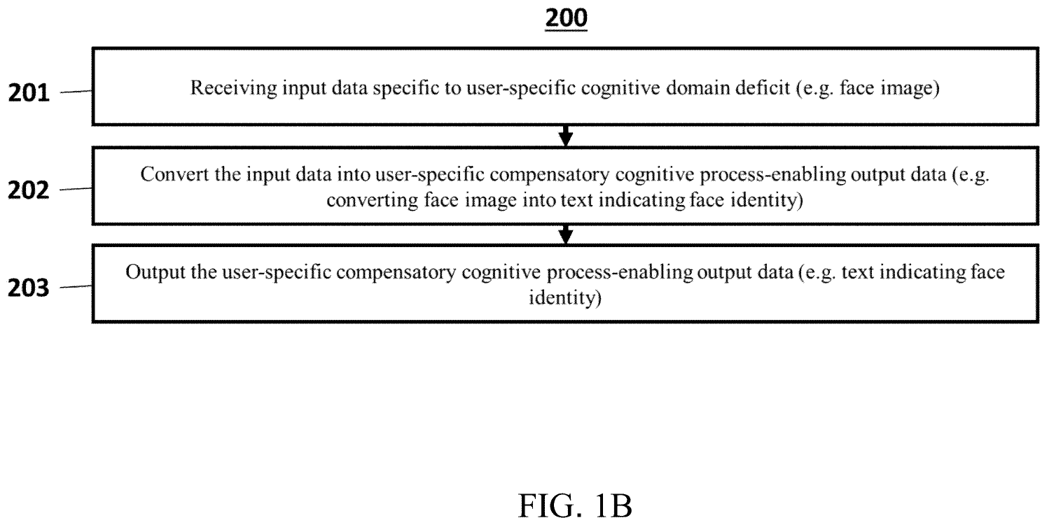

FIG. 1A illustrates an exemplary design of the cognitive assistance system (100). In some embodiments, the system (100) contains three portions, including a sensor portion, a data acquisition and processing portion, and an output portion.

The sensor portion includes one or more optical sensor (101), audio sensor (102), location sensor (103), and other sensors (104). The system (100), in one embodiment, can be a device for dementia cognitive assistance. Other sensors (104), in one embodiment, can be any other sensors or data sources.

The data acquisition and processing portion contains one or more data acquisition module (105), data analysis module (106), and cognitive feedback module (107). The data acquisition module (105), in one embodiment, receives data from the optical sensor (101) audio sensor (102), location sensor (103), and or any other sensors (104). The cognitive feedback module (107), in one embodiment, provides cognitive feedback to the user via a display unit (108), a speaker (109), and/or any other devices (110). The cognitive feedback module (107) can also provide data to a specific information database (113), so the data could be employed to optimize the analysis process of the data analysis module (106).

The output portion contains one or more display unit (108), speaker (109), and other devices (110). Any other devices (110) that can receive data from the cognitive assistance system (100) are within the scope of the Present Specification.

In some embodiments, the sensor portion, the data acquisition and processing portion, and the output portion are in a single machine or device. In some other embodiments, the sensor portion, the data acquisition and processing portion, and the output portion are in a separate machines or devices, such as in a remote smart phone and in a remote server.

FIG. 1B is a flowchart of a method to convert data for cognitive compensation purpose. In some embodiments, part of the method (200) can be executed by using the cognitive assistance system, including the data acquisition and processing portion described above. In one embodiment, it could be executed by the data analysis module.

For automated determination of cognitive assistance data to be presented to the user, method (200) begins at block (201), where the method receives input data necessitating cognitive processing from one or more sensors (e.g., sensor portion described above) through the data acquisition module. In one embodiment, the input data is specific to certain cognitive domain (domain specific). In one embodiment, the input data are data that the user's brain are ineffective in processing. The data may be image data, sound data, text data, location data, behavioral data, activity data, and/or any other data.

At block (202), the method converts the input data into output data that would allow compensatory cognitive process to take place. In one embodiment, the output data would allow the user's part of brain with preserved function could compensate for the ineffective part of brain's function. In one embodiment, the output data could be domain specific. In one embodiment, the system could identify and recognize faces, objects, texts, locations, positions from image data acquired. In one embodiment, the system could identify and recognize language information from the sound data acquired.

At block (203), the method output the converted data. In one embodiment, the methods can transmit data to the cognitive feedback module to provide cognitive assistance to the user. In one embodiment, the system can present the cognitive assistance data output by giving a high presentation priority to data analysis results with a high priority level, which in some embodiments can be allowing the data analysis to have a higher system processing hierarchy, to be presented longer and/or to be presented prominently. In one embodiment, the system can present the cognitive assistance data output by giving low presentation priority to data analysis results with low priority level, which in some embodiments can be allowing the data analysis to have lower system processing hierarchy, to be presented lower, to be presented less prominently, to be ignore by the system, and/or to be omitted altogether.

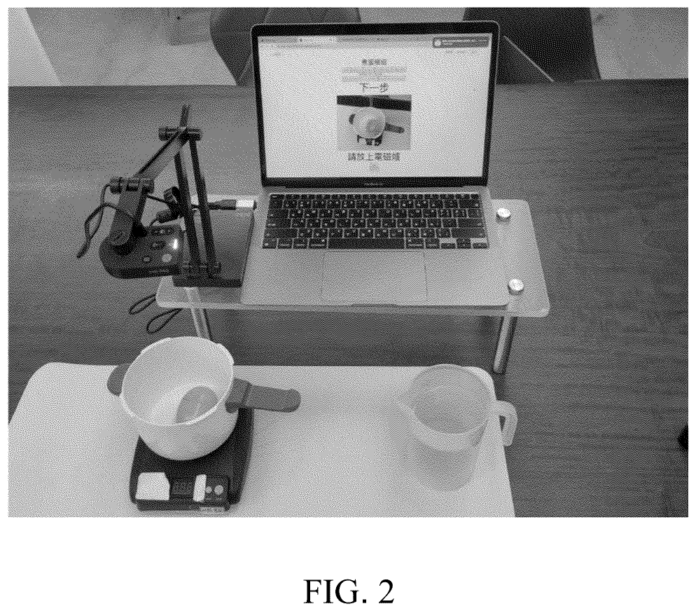

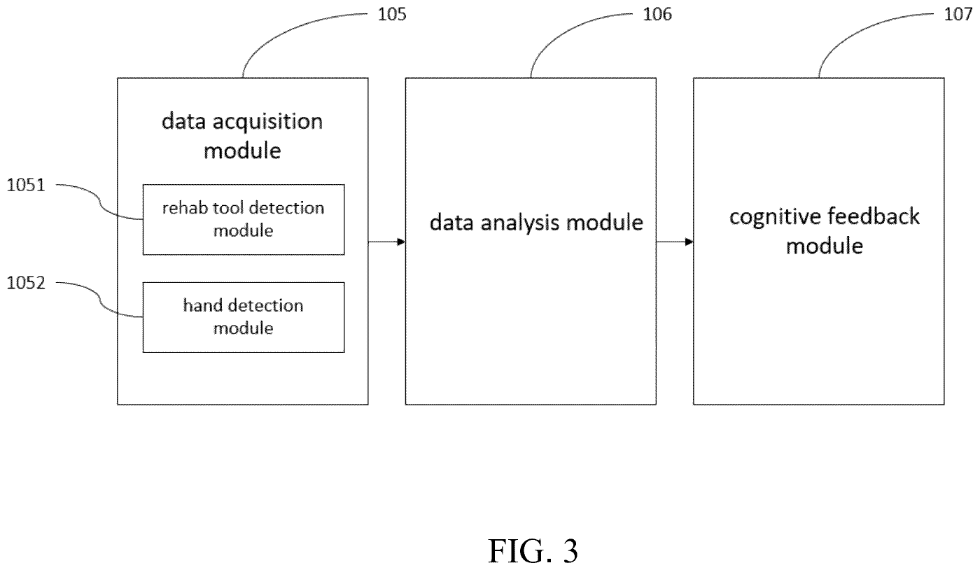

FIG. 2 and FIG. 3 include an example of the system shown in FIG. 1A. In this example, a camera with an adjustable support frame is used as the optical sensor. The data acquisition module (105), data analysis module (106), and cognitive feedback module (107) are software modules installed in a host computer. The display unit is an LCD display connected with the host computer. A set of rehab tools, including a pot, a stove and a water kettle are provided in front of the camera. A white pad is disposed below the rehab tools.

FIG. 3 is an example of the data acquisition module (105), the data analysis module (106) and the data cognitive feedback module (107). The data acquisition module (105) may include object detection modules such as a rehab tool detection module (1051) and a hand detection module (1052). In one of the examples, the rehab tool detection module (1051) may be developed and customized based on Yolo version 5. In one of the examples, the hand detection module (1052) may be developed and customized based on MediaPipe by Google®.

Upon the data acquisition module (105) detects from the images captured by the camera that a user is moving the rehab tools by hand, the data analysis module (106) analyzes whether the user completes the task. For example, the data analysis module (106) would analyze the bounding boxes and labels output from the data acquisition module (105) to see if a specific rehab tool is disposed at a specific position by the hand of the user relative to another rehab tool. Once the bounding boxes and labels show that the relative positions of the rehab tools are correct, the data analysis module (106) determines that the user has completed the task.

In some embodiments, the rehab tool can include a computer vision identifiable tag or label assisting identification and tracking the movement of the rehab tool. The tag or label can be a laser tag, a QR code, or any other computer vision or sensor identifiable elements (such as, RFID tags).

Once the task is completed, the cognitive feedback module (107) provides a task message on the display, such as update the task or update the task completion score. The task message may have different levels of difficulties. For example, the data cognitive feedback module (107) may provide both an image and texts of the next task, which would be easier for the user to understand what should be done. Alternatively, the data cognitive feedback module (107) may provide texts only without an image, which would be more difficult for the user. The data cognitive feedback module (107) may provide different feedback message based on, for example, previous task completion scores of the user, or the setting of the caretaker of the user.

The task completion score may be calculated according to the time spent by the user to complete the task and/or the number of times the user makes mistakes. For example, the user gets 10 points of he or she completes the task without any mistake, 1 point will be deducted every second once the user has spent more than 90 seconds.

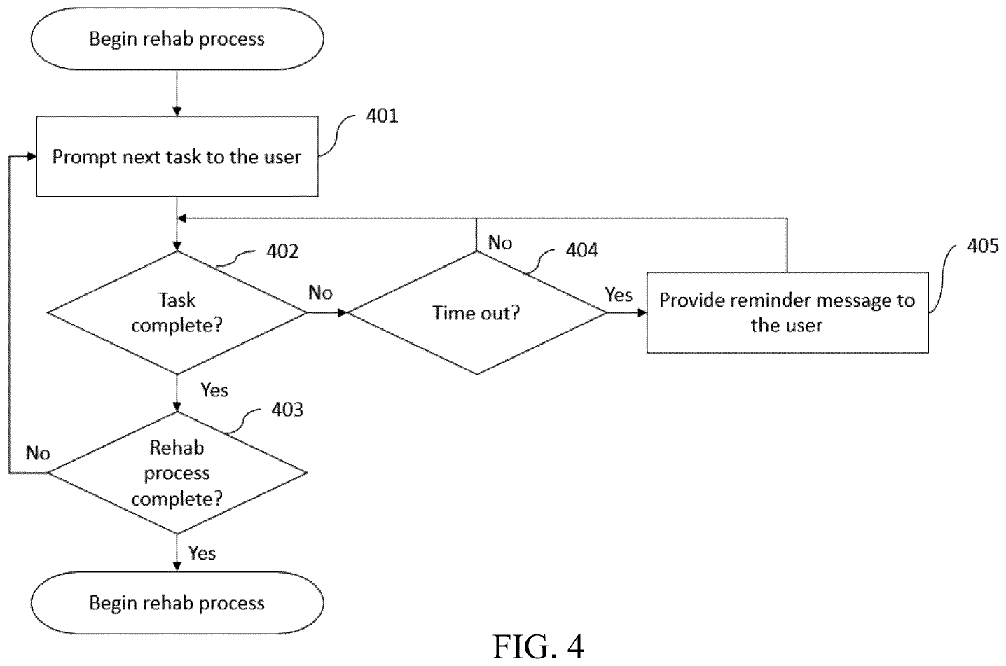

FIG. 4 and FIG. 1A include an exemplary flowchart of the system shown in FIG. 2. In step (401), the system (100) prompts on the display unit (108) a task message of the current task to be completed. Then, in step (402) the data analysis module (106) determines whether the task is completed. If the task is completed, in step (403) the cognitive feedback module (107) determines if the rehab process is completed. If the process is not completed, the process returns to step (401) to prompt the next task message on the display unit (108). If the rehab process is completed, the cognitive feedback module (107) may prompt the rehab result on the display unit (108).

If the data analysis module (106) determines that the task is not completed, it determines if a preset time has passed. If yes, in step (405) the cognitive feedback module (107) provides a reminder message to the user. The reminder message may be an image, a text message, or an audio message related the task.

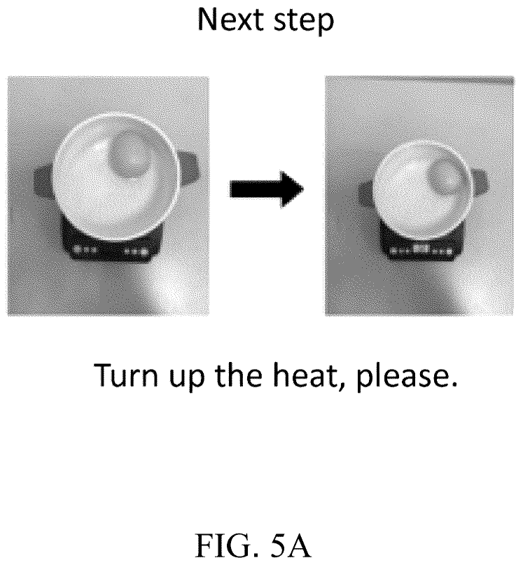

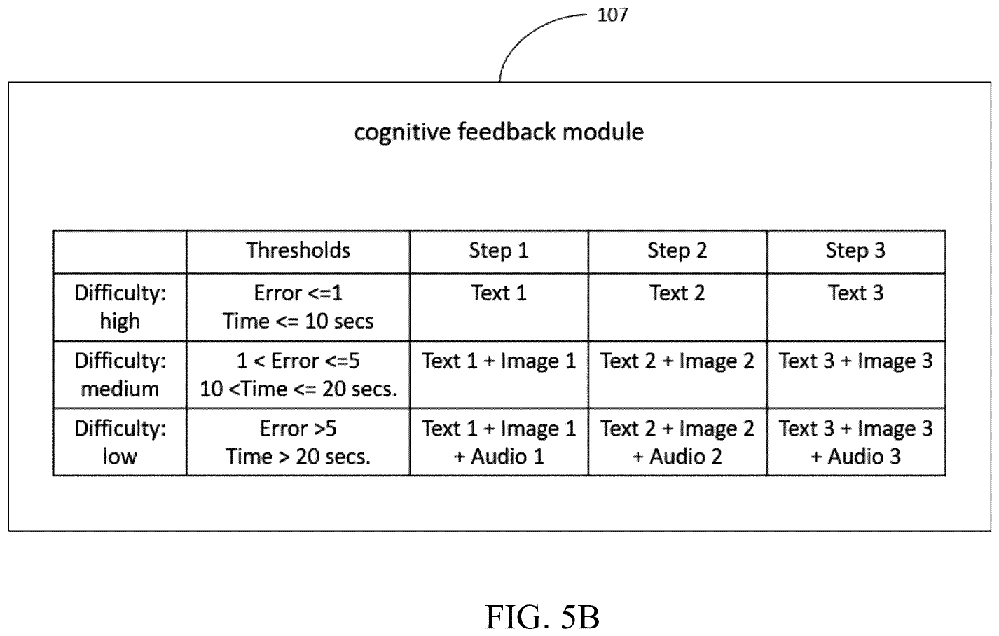

FIG. 5A and FIG. 1A illustrate another exemplary output screen generated by the cognitive assistance system (100) to provide cognitive assistance for executive function. When prompting the task to the user on the display unit (108), the cognitive feedback module (107) may choose to prompt the text message together with an image of the task, or prompt the text only without the image. This would change the difficulty of the task, and the system (100) may adjust the difficulties of different tasks for different users.

FIG. 5B and FIG. 1A include an example of the cognitive feedback module (107). In one embodiment, the cognitive feedback module (107) may dynamically adjust the task difficulty based on the task completion accuracy, the task completion speed, the corresponding Executive Function Performance Test (EFPT) score, or the corresponding Instrumental Activity of Daily Living (IADL) score. This may help improve the attentional control, the cognitive inhibition, the working memory and/or the cognitive flexibility of the user. For example, when the data analysis module (106) determines that the user made an error at step 1, the system (100) may store the number of times the user made the error at step 1. When the number of times exceeds a threshold of 5 times, the cognitive feedback module (107) may prompt a different feedback message with a low difficulty, which includes a text message, an image and an audio message correspond to step 1, to encourage the user to complete the task correctly.

In another example, if the cognitive feedback module (107) prompts a feedback message of medium difficulty including a text message and an image at step 3, and the time spent by the user is only 5 seconds, the cognitive feedback module (107) may adjust the difficulty level to high next time the user performs step 3. Except for adjusting the difficulty levels automatically, the system (100) may also provide an adjustment recommendation to the physician of the user, so that the physician can adjust the difficulty setting manually or a guideline for the commuter system to generate an adjusted treatment program based on the guidelines. For example, once the user finished all steps of a rehab module, the system (100) may provide a report summarizing the numbers of errors made and the times spent at different steps, options to adjust the difficulty levels of the steps, and a difficulty adjustment recommendation based on thresholds shown in FIG. 5B. The physician of the user may adjust the difficulty settings, treatment types, and treatment plans, manually based on the adjustment recommendations provided by the system (100).

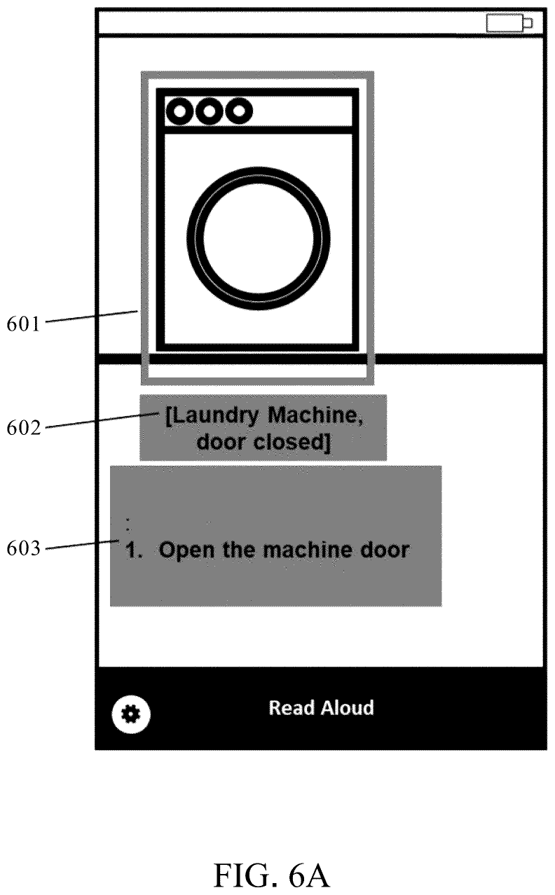

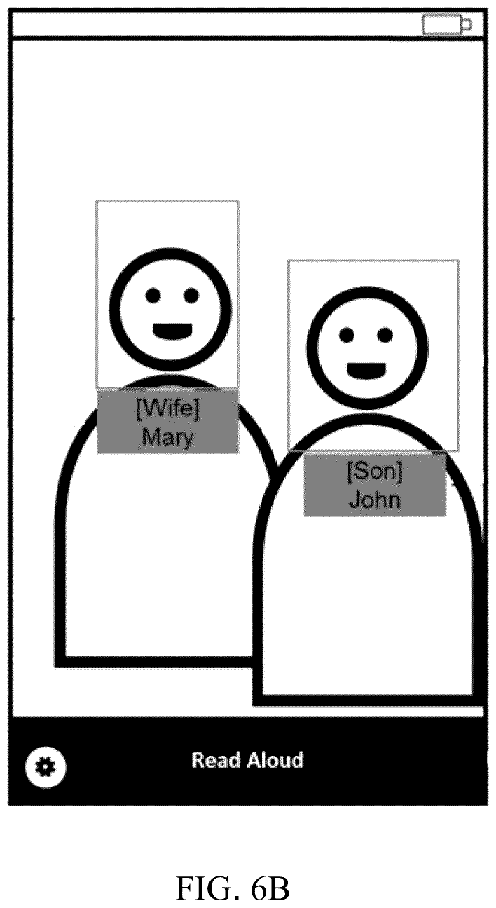

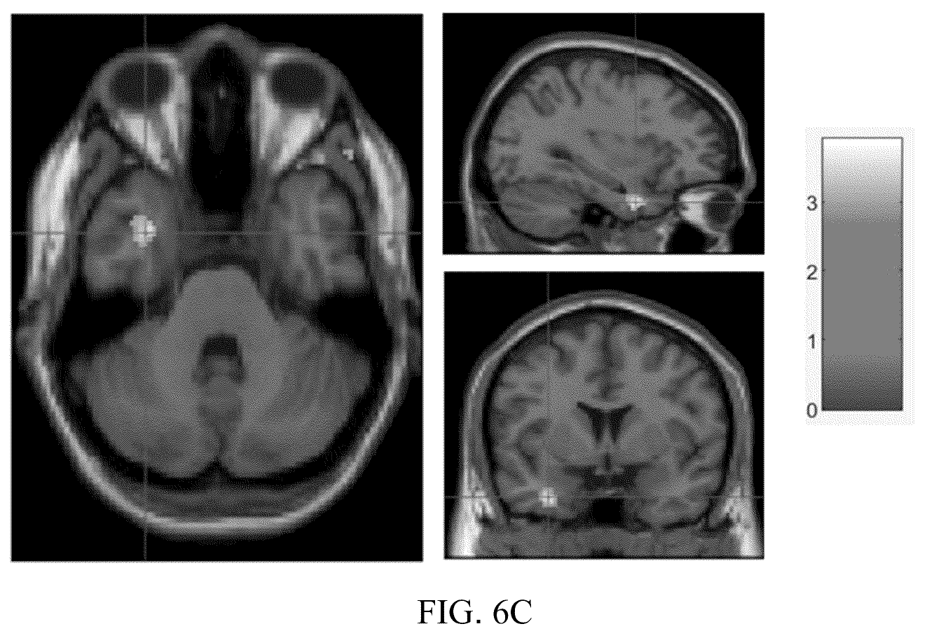

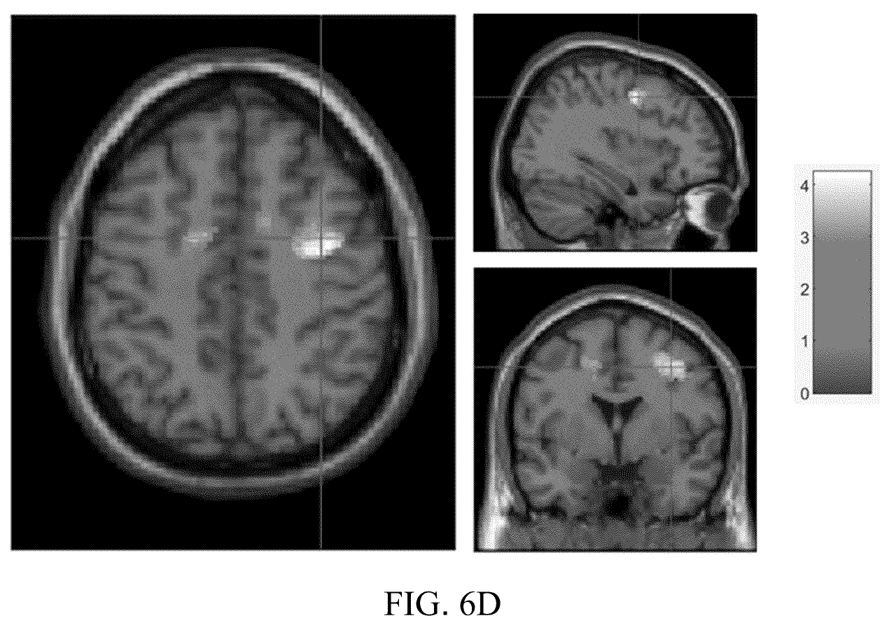

FIG. 6A and FIG. 1A illustrate another exemplary output screen (e.g., the output portion as described above in the FIG. 1A) generated by the cognitive assistance system (100) to provide cognitive assistance for executive function by converting an image showing a certain task state into text data indicative of action that needed to be performed by the user. Conversion of data of image showing a certain task state into text data indicative of action that needed to be performed would be useful to patients with poor executive function but preserved text comprehension function. In this example, the user's brain might be ineffective in executive function and have difficulty determining the next step of action that needed to be performed, so the system receive data of image showing a certain task state as input data necessitating cognitive processing. In this example, the user's brain might still have preserved text comprehension function, so the user could still use the preserved text comprehension function to compensate for the ineffective executive function. In this example, conversion of data of image showing a certain task state into text data indicative of action that needed to be performed would allow compensatory cognitive process recognition to take place, which is allowing the user's text comprehension function to compensate for the ineffective executive function.

In one embodiment, the screen could be generated by the cognitive feedback module (107). In one embodiment of the screen, the system identifies the object on the image (601), and display the information associated with the object (602, 603) on the screen. In one embodiment, it could determine the steps a user would need to perform in order to interact with the object, and determine what steps have already been performed by the user and/or what steps have not been performed by the user. The determination can be done by analysis of image acquired by the data acquisition module, analysis of electronic signals transmitted by the object, or analysis of other data acquired by the system (100). The determination can be based on the analysis of user behavior, which in one embodiment can be based on recognition of user activity captured by one or more sensors.

In one embodiment, the cognitive assistance system (100) provides cognitive assistance for executive function by determining the quality of each step and final result of the task through analysis of the person(s), the object(s), and the interaction(s) in the image. In one embodiment, it could display the quality of the task step or the task. In one embodiment, it could provide instruction to improve the quality of the task, and/or to correct mistake that occurred.

In one exemplary embodiment, a video analysis of the patient's tooth-brushing movement could be done to assess whether the tooth-brushing task is performed correctly. In one exemplary embodiment, an image analysis of the patient's teeth could be done to assess the quality of teeth cleaning.

In one embodiment, the cognitive assistance system (100) provides cognitive assistance for executive function by determining the state of the task through analysis of the person(s), the object(s), and the interaction(s) in the image. It could then provide instruction for the next step of the task to the patient.

In some embodiments, the cognitive assistance system (100) could provide cognitive assistance for tasks such as food preparation, food intake, personal hygiene, bathing, dental clearing, cleaning, dressing, and social tasks.

FIG. 6B and FIG. 1A illustrates an exemplary output screen generated by the system (100) to provide cognitive assistance by converting facial image data into text data. Conversion of facial image data into text data would be useful for patients with poor face memory but preserved text comprehension function. In this example, the user's brain might be ineffective in processing the face image, so the system receives face image as input data necessitating cognitive processing. In this example, the user's brain might still have preserved text comprehension function, so the user could still use the preserved text comprehension function to compensate for the ineffective face memory. In this example, conversion of facial image data into text data would allow compensatory cognitive process recognition to take place, which is allowing the user's text comprehension function to compensate for the ineffective face memory function.

FIG. 6C depicts a functional magnetic resonance imaging of a patient with facial recognition impairment doing a face recognition task without device assistance. The image showed that the right anterior temporal lobe is activated, and demonstrated that the patient uses an ineffective brain area for facial recognition, as illustrated in FIG. 6C.

FIG. 6D depicts a functional magnetic resonance imaging of a patient with facial recognition impairment doing a face recognition task device assistance. The image showed that the precentral gyrus is activated and demonstrated that the patient uses the text comprehension area for facial recognition, bypassing the ineffective brain area for face recognition, as illustrated in FIG. 6C.

In one study, eight patients (5 females and 3 males) with dementia were enrolled in this study. The mean age of the healthy subjects was 76.87 years±9.67. A mean Mini-mental state examination (MMSE) score of 20.87±4.05 and Clinical Dementia Rating (CDR) score of 0.81±0.53 suggest that those patients have mild dementia. The mean score of 15.87±11.62 on the Dementia Severity Rating Scale (DSRS) also indicates that enrolled patients have mild difficulty completing tasks in daily life. The Activity of Daily Activity (ADL) score of 87.50±16.903 and IADL score of 10 confirmed impaired daily activity as well.

All the patients were evaluated with EFPT in the study. The mean EFPT score without device assistance was 4.50±5.318 and the mean EFPT score with device assistance was 2.63±5.854 (p<0.05), which has successfully validated the efficacy of executive function cognitive prosthesis in assisting patients having mild dementia with food preparation. The patient's task completion test data is shown in Table 1:

TABLE 1

| | Without device assistance | | With device assistance | |
|---|---|---|---|---|
| | | Task | | Task |
| | Time | completion | | completion |
| | taken (s) | score | Time taken (s) | score |
| Step 1 | 19.00 ± 25.77 | 1.63 ± 0.74 | 16.00s ± 27.23 | 1.88 ± 0.35 |
| Step 2 | 15.75 ± 14.94 | 1.63 ± 0.74 | 10.13 ± 7.00 | 1.88 ± 0.35 |
| Step 3 | 9.00 ± 10.77 | 1.88 ± 0.35 | 16.88 ± 17.23 | 1.88 ± 0.35 |
| Step 4 | 17.38 ± 12.19 | 2.00 ± 0.00 | 21.00 ± 18.69 | 1.75 ± 0.71 |
| Step 5 | 39.00 ± 35.57 | 1.63 ± 0.74 | 11.38 ± 7.41* | 1.75 ± 0.71 |
| Step 6 | 20.63 ± 5.04 | 1.75 ± 0.71 | 24.38 ± 9.12 | 1.75 ± 0.71 |
| Total | 135.50 ± 97.80 | 10.50 ± 2.15 | 121.38 ± 87.26 | 10.88 ± 1.64 |

*p < .05,
** p < .01,
*** p< .001, n = 8

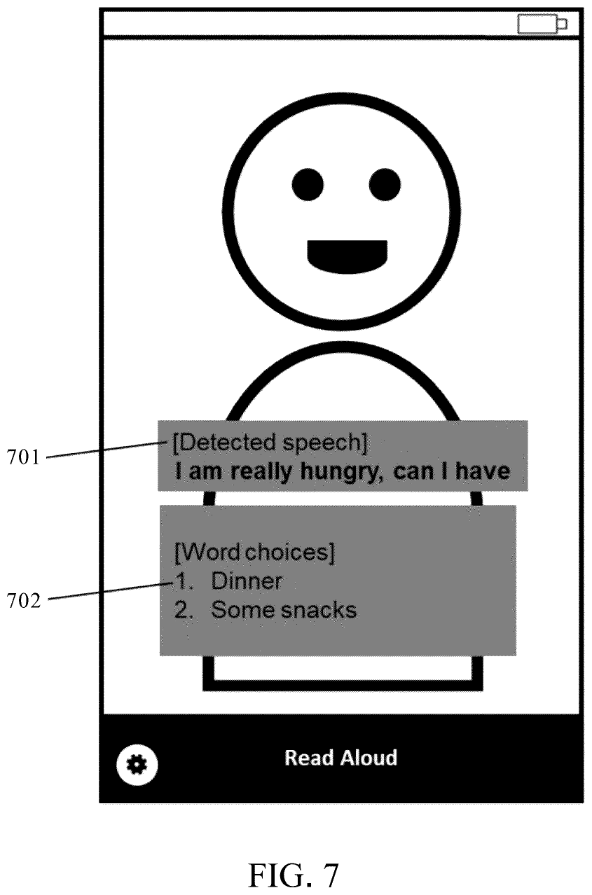

FIG. 7 and FIG. 1A illustrate an exemplary output screen generated by the cognitive assistance system (100) to provide cognitive assistance related to language use by converting detected speech data into text data indicative of potential word choices that could be used by the user. Conversion of speech data into text data indicative of potential word choices would be useful to patients with ineffective speech generation function but preserved text comprehension function. In this example, the user's brain might be ineffective in speech generation, so the system receives speech data as input data necessitating cognitive processing. In this example, the user's brain might still have preserved text comprehension function, so the user could still use the preserved text comprehension function to compensate for the ineffective speech generation function. In this example, conversion of speech data would allow compensatory cognitive process recognition to take place, which is allowing the user's text comprehension function to compensate for the ineffective speech generation function.

In one embodiment, the screen could be generated by the cognitive feedback module (107). In one embodiment of the screen, the screen analyzes the speech it captured through an audio sensor (102), and display the result of speech analysis (701). In one embodiment, it could determine possible words a user would need to use to converse (702) by using a computing device or AI (artificial intelligence). The determination can be done by the analysis of voice acquired by the data acquisition module, the analysis of language content through natural language processing.

In one embodiment, the image could be acquired by the data acquisition module (105) through the audio sensor (102), analyzed by the data analysis module (106), and then displayed together with data analysis results, by the cognitive feedback module (107) through the display unit (106). The data analysis results displayed, in one embodiment, can be based on the priority level determined by the system based on specific information data stored in the specific information database (113). For example, speech recognition results involving a word specified in the specific information database to have a high frequency of prior use by the user may be given a higher presentation priority.

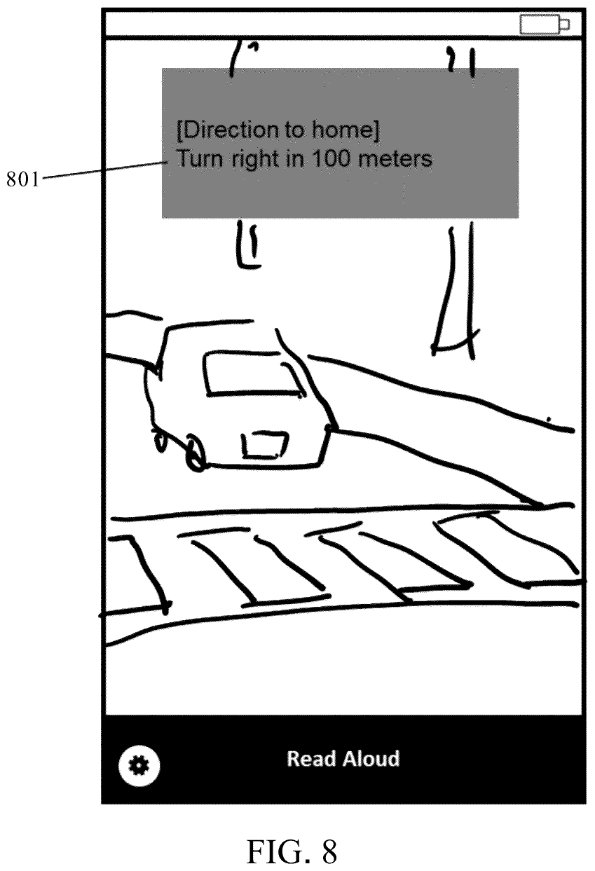

FIG. 8 and FIG. 1A illustrate an exemplary output screen generated by the cognitive assistance system (100) to provide cognitive assistance related to navigation by converting detected environmental data into text data indicative of direction for the user to reach the destination. Conversion of environmental data into text data indicative direction would be useful to patients with ineffective spatial navigation function but preserved text comprehension function. In this example, the user's brain might be ineffective in spatial navigation, so the system receives environmental data as input data necessitating cognitive processing. In this example, the user's brain might still have preserved text comprehension function, so the user could still use the preserved text comprehension function to compensate for the ineffective spatial navigation function. In this example, conversion of environmental data would allow compensatory cognitive process recognition to take place, which is allowing the user's text comprehension function to compensate for the ineffective spatial navigation function.

In one embodiment, the screen could be generated by the cognitive feedback module (107). In one embodiment of the screen, the system analyzes the location data captured through a sensor (102) and determines the user's current location. In one embodiment, it could determine possible directions the user need to be heading to (801). The determination can be done by analysis of location data by the data acquisition module, analysis of planned activity data stored in the specific information database (111), analysis of past location data stored in the specific information database (113), or analysis of past behavioral data stored in the specific information database (113).

In one embodiment, the data could be acquired by the data acquisition module (105) through the location sensor (103), analyzed by the data analysis module (106). In one embodiment, the data analysis results could be displayed by the cognitive feedback module (107) through the display unit (106). The data analysis results displayed, in one embodiment, can be based on the priority level determined by the system based on specific information data stored in the specific information database (113). For example, route navigation results involving a route specified as a destination in the pre-specified schedule stored in the specific information database, on the scheduled time, may be given a higher presentation priority.

The data acquisition and proceeding portion as described herein can use the data acquired to generate a computer-generated treatment plan (e.g., generative AI for generating a treatment plan), which uses the data acquired (e.g., electronic signal) through the sensors to optimize the formula for an optimized treatment plan.

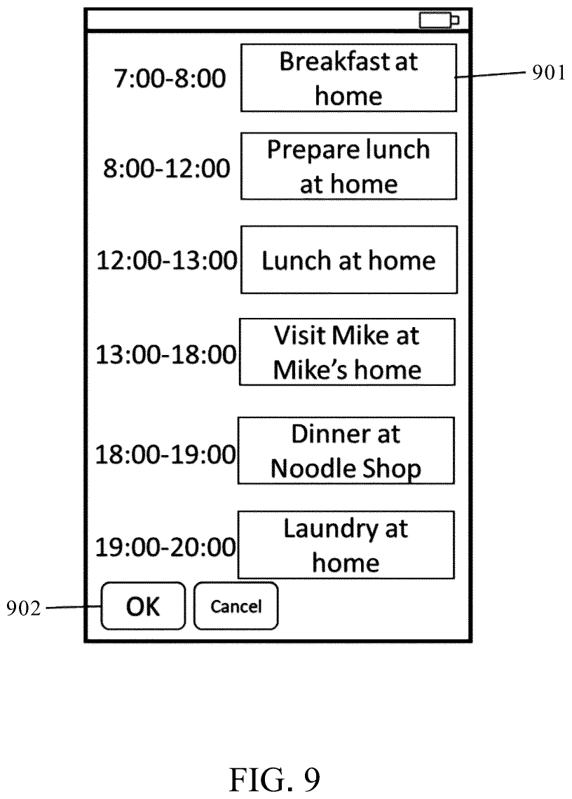

FIG. 9 and FIG. 1A illustrate an exemplary schedule setting screen of the cognitive assistance system (100). In one embodiment, a user could specify a pre-planned daily schedule via the screen. In the embodiment shown, a user can specify a planned activity for a specific time by tapping on an activity button (901). In one embodiment, a user can specify the event, time, location, and person associated with the activity. A user can also save the planned activity into the specific information database by tapping the “ok” button (902) on the screen.

In one embodiment, the schedule stored in the specific information database (113) could modify the output of the data analysis module (106) to provide optimized cognitive assistance in accordance with the user's needs.

In some embodiments, the cognitive assistance system (100) could provide cognitive assistance to improve the patient's attention, judgement, calculation, memory, social, and/or language functions.

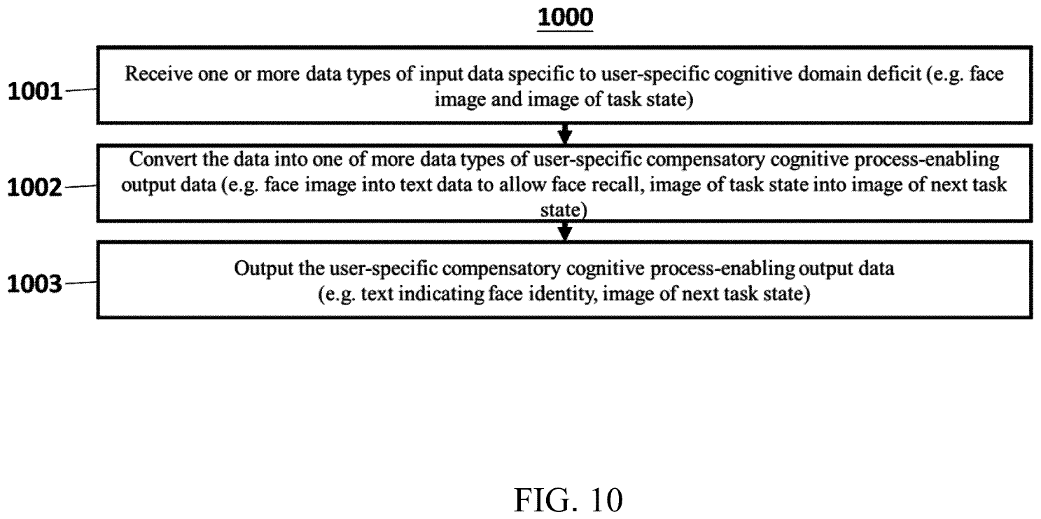

FIG. 10 and FIG. 1A illustrate a flowchart of a method to convert one or more data type into one or more data type for cognitive compensation purpose. In some embodiments, part of the method (1000) can be executed using the cognitive assistance system (100). In one embodiment, it could be executed by the data analysis module (106).

For automated determination of cognitive assistance data to be presented to the user, the method (1000) begins at block (1001), where the method receives input data necessitating cognitive processing from one or more sensors through the data acquisition module (105). In one embodiment, the input data is could be for multiple cognitive domains. In one embodiment, the input data, are data that the user's brain are ineffective in processing. The data may be image data, sound data, text data, location data, behavioral data, activity data, and/or any other data.

At block (1002), the method could convert the input data into output data that would allow one or more compensatory cognitive process to take place. In one embodiment, the output data would allow the user's multiple part of brain with preserved function could compensate for the ineffective part of brain's function. In one embodiment, the output data could be for multiple domains. In one embodiment, the system could identify and recognize faces, objects, texts, locations, positions from image data acquired. In one embodiment, the system could identify and recognize language information from the sound data acquired.

At block (1003), the output the converted data. In one embodiment, the methods can transmit data to the cognitive feedback module (107) to provide cognitive assistance to the user.

In one embodiment, the system can present the cognitive assistance data output by giving a high presentation priority to data analysis results with a high priority level, which in some embodiments can be allowing the data analysis to have a higher system processing hierarchy, to be presented longer and/or to be presented prominently. In one embodiment, the system can present the cognitive assistance data output by giving low presentation priority to data analysis results with low priority level, which in some embodiments can be allowing the data analysis to have lower system processing hierarchy, to be presented lower, to be presented less prominently, to be ignore by the system, and/or to be omitted altogether.

In one embodiment, the cognitive assistance system (100) could provide visual and/or audio reward when desired behavior is achieved by the patient, to encourage patient compliance toward the cognitive assistance.

In one embodiment, the cognitive assistance system (100) would automatically optimize user interface elements based on the patient's status. In one embodiment, the cognitive assistance system (100) would automatically optimize user interface elements based on the caregiver's status.

In some embodiments, the cognitive assistance system (100) would track, report, and/or analyze the following data: care cost saved, caregiver time saved, interruption requiring caregiver intervention, caregiver satisfaction, patient satisfaction, patient functional performance, patient rehabilitation performance, patient psychological status (e.g. mood, confidence), patient behavioral changes.

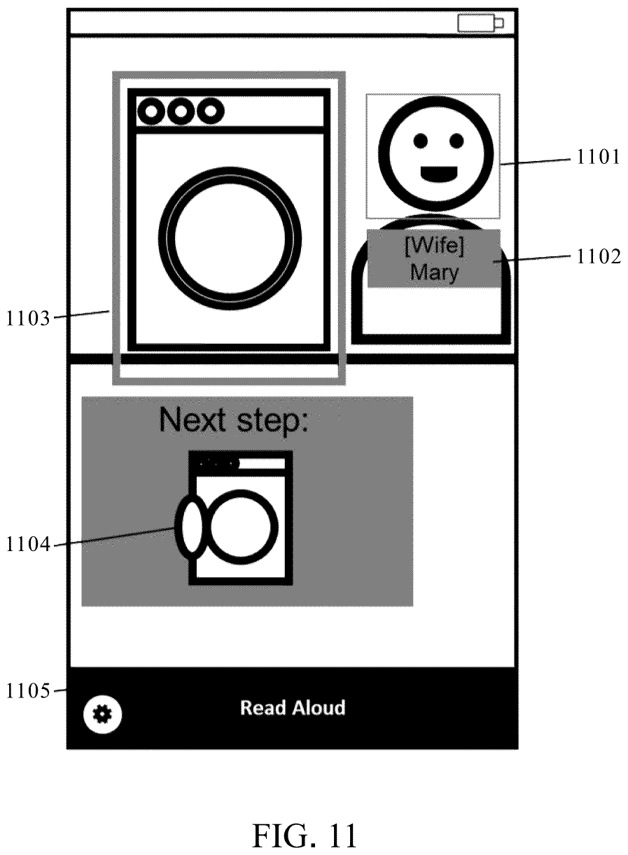

FIG. 11 and FIG. 1A illustrate an exemplary output screen generated by the cognitive assistance system (100) to provide cognitive assistance by converting facial image data into text data and converting an image showing a certain task state into text data indicative of action that needed to be performed by the user. Conversion of facial image data into text data would be useful for patients with poor face memory but preserved text comprehension function, and conversion of data of image showing a certain task state into text data indicative of action that needed to be performed would be useful to patients with poor executive function but preserved text comprehension function. In this example, the user's brain can be ineffective in processing the face image and have difficulty determining the next step of action that needed to be performed, so the system receive face image and data of image showing a certain task state as input data necessitating cognitive processing. In this example, the user's brain might still have preserved text comprehension function, so the user could still use the preserved text comprehension function to compensate for the ineffective face memory and executive function. In this example, conversion of facial image data into text data would allow compensatory cognitive process recognition to take place, which is allowing the user's text comprehension function to compensate for the ineffective face memory function and executive function.

In one embodiment, the screen could be generated by the cognitive feedback module (107). In one embodiment of the screen, the system identifies the face on the image (1101), and display the text information associated with the face (1102). In one embodiment of the screen, the system converts face image data on the image (1101), and display the text information associated with the face (1102). In one embodiment of the screen, the system identifies the object on the image (1103), and display the information associated with the object (1104, 1105) on the screen. In one embodiment, it could determine the steps a user would need to perform in order to interact with the object, and determine what steps have already been performed by the user and/or what steps have not been performed by the user. The determination can be done by analysis of image acquired by the data acquisition module, analysis of electronic signals transmitted by the object, or analysis of other data acquired by the system (100).

In one embodiment, the image could be acquired by the data acquisition module (105) through the optical sensor (101), analyzed by the data analysis module (106), and then displayed together with data analysis results, by the cognitive feedback module (107) through the display unit (106).

In one embodiment, a user can also obtain the audio data analysis result through a speaker (109), by tapping on a button (303) on the screen. This would allow the converting facial image data into audio data, which would be useful for patients with poor face memory and executive function but preserved speech comprehension function.

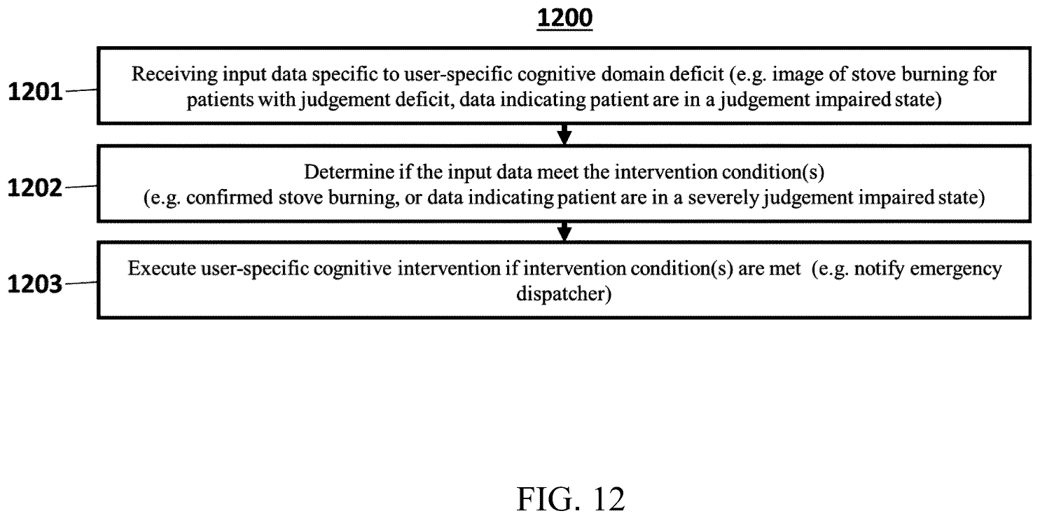

FIG. 12 and FIG. 1A illustrate a flowchart of a method related to execution of cognitive intervention. In some embodiments, part of the method (1300) can be executed using the cognitive assistance system (100). In one embodiment, it could be executed by the data analysis module (106).

For automated determination of cognitive assistance data to be presented to the user, the method (1200) begins at block (1201), where the method receives input data (specific to user-specific cognitive domain deficit) from one or more sensors through the data acquisition module (105). In one embodiment, the input data is specific to certain cognitive domain (domain specific). In one embodiment, the input data, are data that the user's brain are ineffective in processing. The data may be image data, sound data, text data, location data, behavioral data, activity data, and/or any other data.

At block (1202), the method determines if the data meet the intervention condition(s). In one embodiment, the output data would allow the user's part of brain with preserved function could compensate for the ineffective part of brain's function. In one embodiment, the output data could be domain specific. In one embodiment, the system could identify and recognize faces, objects, texts, locations, positions from image data acquired. In one embodiment, the system could identify and recognize language information from the sound data acquired.

At block (1203), the method executes user-specific cognitive intervention if intervention condition(s) are met. In one embodiment, the methods can transmit data to the cognitive feedback module (107) to execute the cognitive intervention.

In one exemplary embodiment, after the method received an image of burning stove, the method confirmed that image met one of the intervention conditions, which require fire to present in the image. In the exemplary embodiment, the method could then notify emergency dispatcher.

In one exemplary embodiment, after the method received data indicating patient are in a judgement impaired state, the method confirmed that the patient's judgement score indeed met one of the intervention conditions, which require judgement score fall within a certain range. In the exemplary embodiment, the method could then notify emergency dispatcher.

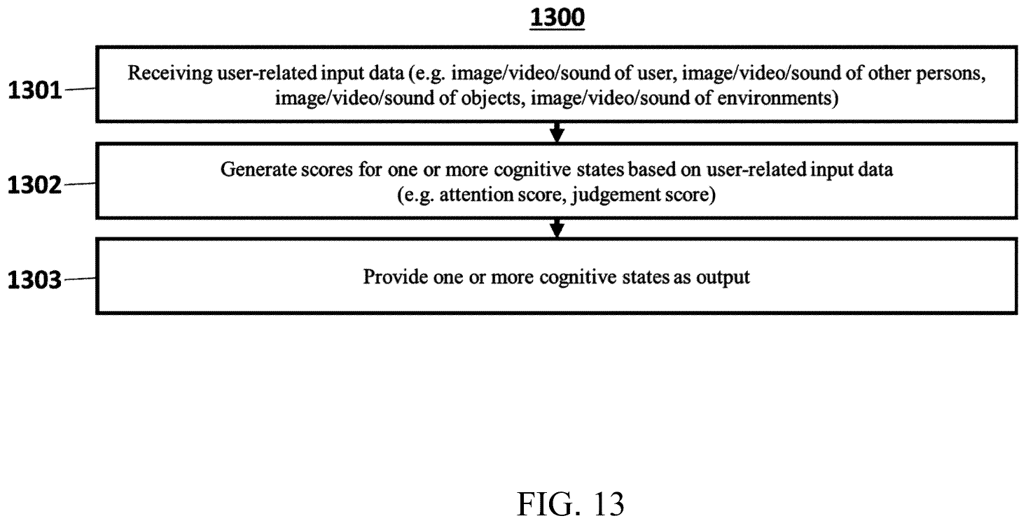

FIG. 13 and FIG. 1A illustrate a flowchart of a method related to cognitive monitoring. In some embodiments, part of the method (1300) can be executed using the cognitive assistance system (100). In one embodiment, it could be executed by the data analysis module (106).

For automated determination of cognitive assistance data to be presented to the user, the method (1300) begins at block (1301), where the method receives user related input data from one or more sensors through the data acquisition module (105). In one embodiment, the input data may be related to user, other person(s), object(s), and/or environment. The data may be image data, sound data, text data, location data, behavioral data, activity data, and/or any other data.

At block (1302), the method could generate scores for various cognitive states based on user-related input data. In one embodiment, the system could identify and recognize faces, objects, texts, locations, positions from image data acquired. In one embodiment, the system could identify and recognize language information from the sound data acquired. In one embodiment, the cognitive state score generated could be attention score indicative of the user's attention status and/or judgement score indicative of the user's judgement status.

At block (1303), the method could provide one or more cognitive states as output. In one embodiment, the methods can transmit data to the cognitive feedback module (107) to execute the cognitive intervention.

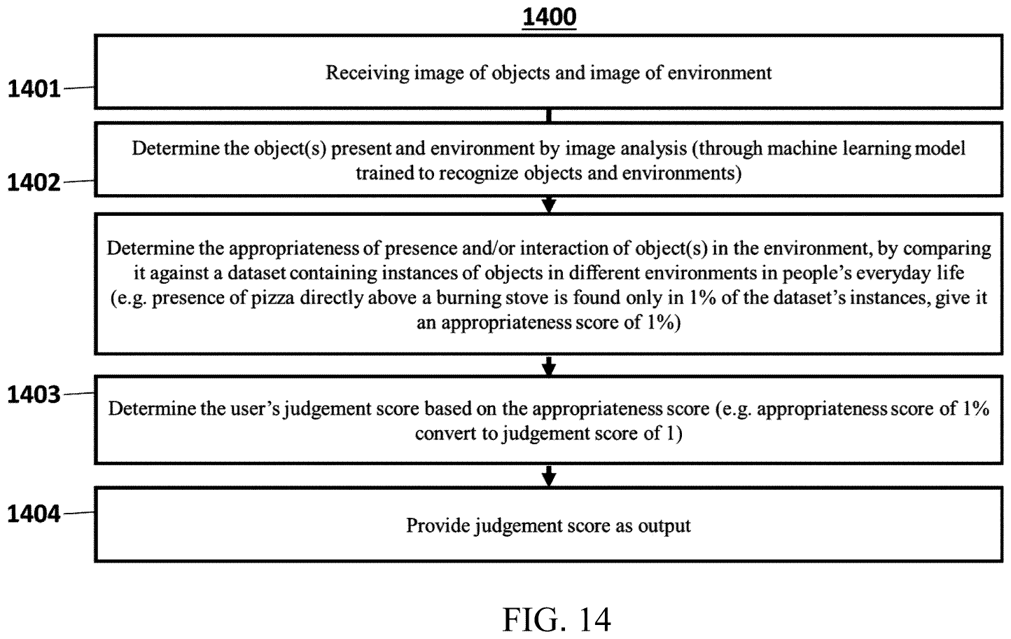

FIG. 14 and FIG. 1A illustrate a flowchart (1400) of an exemplary embodiment of method related to cognitive monitoring (1300). In this embodiment, the method determines the judgement score. In some embodiments, part of the method (1400) can be executed using the cognitive assistance system (100). In one embodiment, it could be executed by the data analysis module (106).

For automated determination of judgement score, the method (1400) begins at block (1401), where the method receives user related input data from one or more sensors through the data acquisition module (105). In one embodiment, the input data may be related to user, other person(s), object(s), and/or environment. The data may be image data, sound data, text data, location data, behavioral data, activity data, and/or any other data.

At block (1402), the method could determine the object(s) present and environment by image analysis. In one embodiment, the determination may be made through machine learning model trained to recognize objects and environments.

At block (1403), the method determines the appropriateness of presence and/or interaction of various object(s) in the environment, by comparing it against a dataset containing instances of objects in different environments in people's everyday life. In an exemplary embodiment, presence of pizza directly above a burning stove is found only in 1% of the dataset's instances, give it an appropriateness score of 1%.

At block (1404), the method determines the user's judgement score based on the appropriateness score. In an exemplary embodiment, an appropriateness score of 1% could be convert to judgement score of 1.

At block (1405), the method could provide the judgement score as output. In one embodiment, the methods can transmit data to the cognitive feedback module (107) to execute the cognitive intervention.

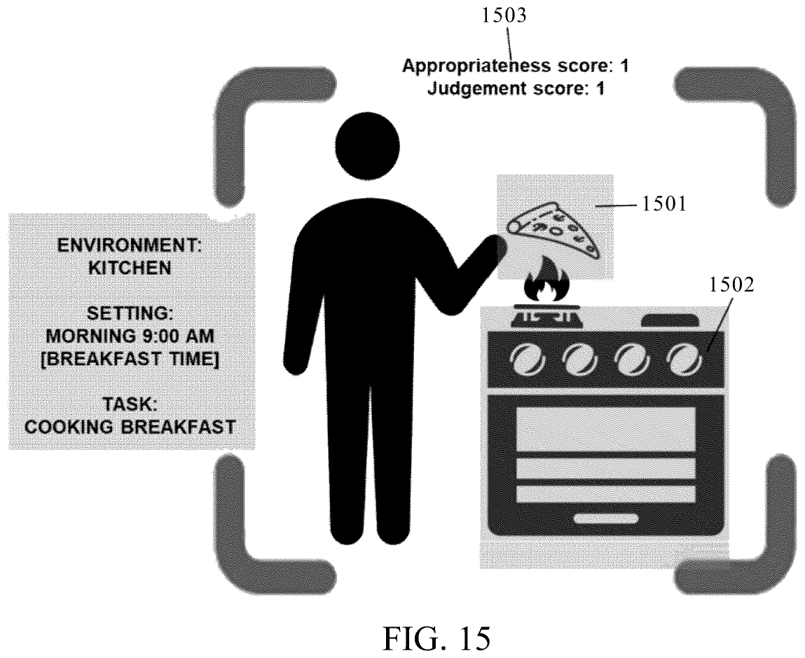

FIG. 15 and FIG. 1A illustrate an exemplary output screen generated by the cognitive assistance system (100) for cognitive monitoring. In one embodiment, the screen could be generated by the cognitive feedback module (107). In one embodiment of the screen, the system analyzes the object(s) and environment through a sensor (102).

In one embodiment, the data could be acquired by the data acquisition module (105) through the location sensor (103), analyzed by the data analysis module (106). In one embodiment, the data analysis results could be displayed by the cognitive feedback module (107) through the display unit (106).

In one embodiment, the object(s) present and environment are determined by image analysis. In one embodiment, the determination may be made through machine learning model trained to recognize objects and environments. In one exemplary embodiment, the system may detect the presence of pizza (1501) and turned-on stove (1502) in the kitchen through image analysis.

In one embodiment, the appropriateness score(s) are determined based on appropriateness of presence and/or interaction of various object(s) in the environment. In one exemplary embodiment, the appropriateness score is determined by comparing the presence and/or interaction of various object(s) in the environment against a dataset containing instances of objects in different environments in people's everyday life. In an exemplary embodiment, presence of pizza directly above a burning stove is found only in 1% of the dataset's instances, give it an appropriateness score of 1%.

In an exemplary embodiment, an appropriateness score of 1% are converted to judgement score of 1. The system could then provide judgement score of 1 as output (1503)

In one embodiment, the cognitive assistance system (100) could perform analysis on the image and/or sound acquired. In one embodiment, the analysis would determine information about person(s) present, object(s) present, interaction(s) present. Personal information that could be determined including identity, psychological status (e.g. mood, behavior, and/or confidence), cognitive functioning (e.g. attention, judgement, calculation, memory, navigation, social, sanguage, and/or executive function) disease diagnosis, and/or disease status.

In one embodiment, the analysis would determine the presence of threat.

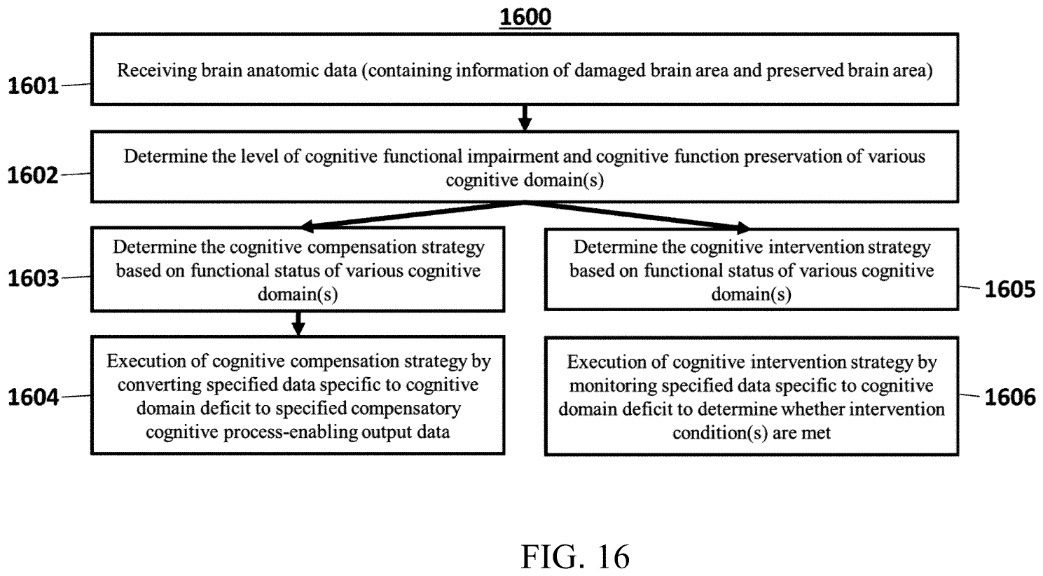

FIG. 16 and FIG. 1A illustrate a flowchart of a method related to execution of cognitive intervention. In some embodiments, part of the method (1300) can be executed using the cognitive assistance system (100). In one embodiment, it could be executed by the data analysis module (106).

For automated determination of cognitive assistance data to be presented to the user, the method (1600) begins at block (1601), where the method receives brain anatomic data through the data acquisition module (105). In one embodiment, the data containing information of damaged brain area and preserved brain area. The data may be magnetic resonance imaging data, functional magnetic resonance imaging data, computed tomography data, positron emission tomography data, image data, text data, location data, and/or any other data.

At block (1602), the method could determine the level of cognitive functional impairment and cognitive function preservation of various cognitive domain(s). In one embodiment, the method could determine level of functioning of specific cognitive domain based on the degree of damage observed on brain anatomic data. The data on whether functioning of specific cognitive domain are preserved or damaged could be used to determine the cognitive compensation strategy (proceed to block (1603)) and/or intervention strategy (proceed to block (1605)).

At block (1603), the method could determine the cognitive compensation strategy based on functional status of various cognitive domain(s). In one embodiment, preserved cognitive function are employed to compensate impaired cognitive function.

At block (1604), the method could execute the cognitive compensation strategy by converting specified data specific to cognitive domain deficit to specified compensatory cognitive process-enabling output data.

At block (1605), the method could determine the cognitive intervention strategy based on functional status of various cognitive domain(s). In one embodiment, impaired cognitive functions are monitored to determine whether intervention is necessary, to avoid danger and/or to improve quality of life.

At block (1606), the method could execute the cognitive intervention strategy by monitoring specified data specific to cognitive domain deficit to determine whether intervention condition(s) are met.

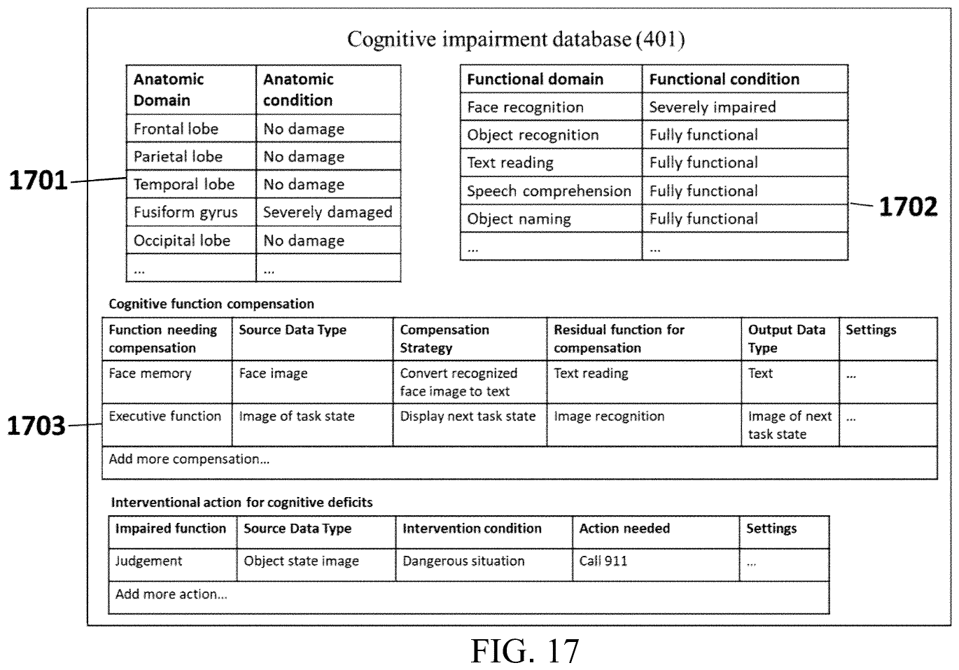

FIG. 17 and FIG. 1A illustrate an exemplary output screen generated by the cognitive assistance system (100) for determination of compensation strategy and/or intervention strategy. In one embodiment, the screen could be generated by the cognitive feedback module (107).

In one embodiment, the data could be acquired by the data acquisition module (105), analyzed by the data analysis module (106). In one embodiment, the data analysis results could be displayed by the cognitive feedback module (107) through the display unit (106).

In one embodiment, the brain anatomic data could be analyzed. In one embodiment, the brain anatomic data and its analysis result showed whether each anatomic part of brain is damaged or preserved (at block (1701)). In one embodiment, the brain anatomic data and its analysis results can also be adjusted by human input.

In one embodiment, the brain functional data could be determined. In one embodiment, the brain functional data and its analysis result are shown (at block (1702)). In one embodiment, the level of functioning of specific cognitive domain could be determined based on the degree of damage observed on brain anatomic data, with brain area with more anatomical damage have higher possibility of having impaired functioning of cognitive function it's responsible for. In one embodiment, the brain functional data and its analysis results can also be adjusted by human input.

In one embodiment, the cognitive compensation strategy could be determined. In one embodiment, the brain compensation strategy is shown (at block (1703)). In one embodiment, the cognitive compensation strategy could be determined based on the level of functioning of specific cognitive domain. In one embodiment, preserved cognitive function are employed to compensate impaired cognitive function, when determining the cognitive compensation strategy. In one embodiment, the cognitive compensation strategy can also be adjusted by human input. In one embodiment, the cognitive assistance system (100) could execute the specified cognitive compensation strategy (at block (1703)) by converting specified data specific to cognitive domain deficit to specified compensatory cognitive process-enabling output data through specified compensation strategy.

In one embodiment, the cognitive intervention strategy could be determined. In one embodiment, the brain intervention strategy is shown (1704). In one embodiment, the cognitive intervention strategy could be determined based on the level of functioning of specific cognitive domain, impaired cognitive function are monitored to determine whether intervention is necessary, to avoid danger and/or to improve quality of life, when determining cognitive intervention strategy. In one embodiment, the cognitive compensation strategy can also be adjusted by human input. In one embodiment, the cognitive assistance system (100) could execute the specified cognitive intervention strategy (1704) by monitoring specified data specific to cognitive domain deficit to determine whether intervention condition(s) are met.

In one embodiment, the cognitive assistance system (100) could assist in determining diagnosis and follow up of neuropsychiatric disease by analyzing the patient's functional status, anatomic lesion, and disease data.

In one embodiment, the cognitive assistance system (100) could suggest a specific treatment strategy based on its analysis of the patient data.

In one embodiment, the cognitive assistance system (100) could suggest a specific behavioral intervention strategy to achieve specific behavioral changes, based on its analysis of the patient data.

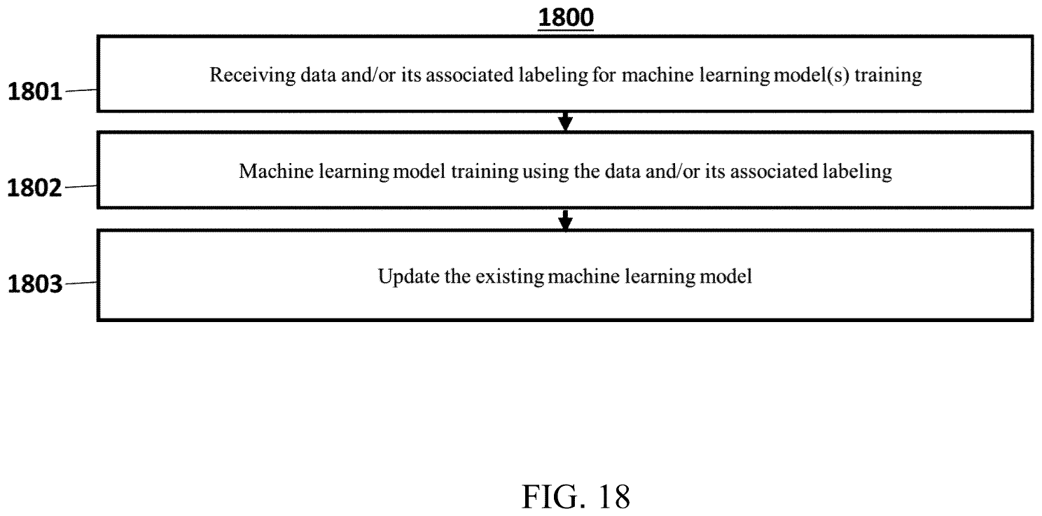

FIG. 18 and FIG. 1A illustrate a flowchart of a method related to update of algorithm for the cognitive assistance system. In some embodiments, part of the method (1300) can be executed using the cognitive assistance system (100). In one embodiment, it could be executed by the data analysis module (106). In one embodiment, it could be used to update the algorithm for data conversion, condition triggering, determination of cognitive state, determination of compensation strategy, and determination of intervention strategy.

For automated determination of cognitive assistance data to be presented to the user, the method (1800) begins at block (1801), where the method receives data and/or its associated labeling for machine learning model(s) training. The data may be data used for cognitive compensation, cognitive intervention, and/or any other data. The data may be data utilized in any steps throughout cognitive compensation process and/or cognitive intervention process.

At block (1802), the method machine learning model training using the data and/or its associated labeling.

At block (1803), the method could update the existing machine learning model. In one embodiment, the method allows the machine learning model(s) to better suit for user-specific needs. In one embodiment, the method allows the machine learning model(s) to have better performance.

In one embodiment, the cognitive assistance system (100) could train machine learning model by utilizing both existing machine learning model and individualized patient data. In one embodiment, the system could transfer the training model and/or data between remote server and local device to achieve specific outcomes for specific machine learning model(s).

In one embodiment, the cognitive assistance system (100) would have a machine learning data and model management system that manages machine learning model(s) and/or training data with its associated labelling.

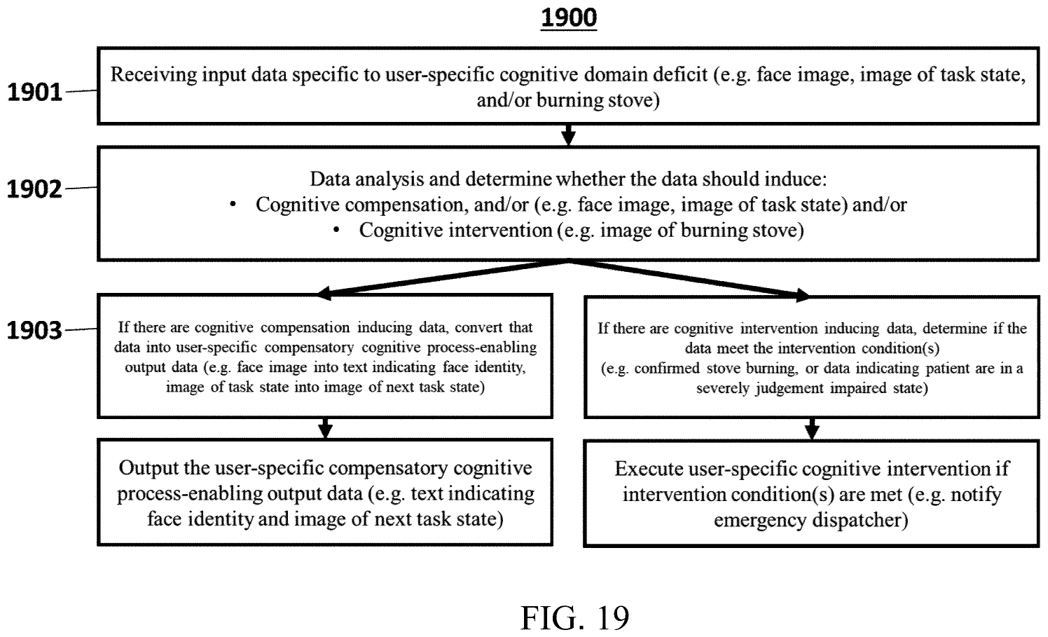

FIG. 19 and FIG. 1A illustrate a flowchart of a method to convert data for cognitive compensation purpose and for execution of cognitive intervention. In some embodiments, part of the method (1900) can be executed using the cognitive assistance system (100). In one embodiment, it could be executed by the data analysis module (106).

For automated determination of cognitive assistance data to be presented to the user, the method (1900) begins at block (1901), where the method receives input data necessitating cognitive processing from one or more sensors through the data acquisition module (105). In one embodiment, the input data is specific to certain cognitive domain (domain specific). In one embodiment, the input data, are data that the user's brain are ineffective in processing. The data may be image data, sound data, text data, location data, behavioral data, activity data, and/or any other data.

At block (1902), the method could analyze the data and determine whether the data should induce cognitive compensation (proceed to block (1903)), and/or cognitive intervention (proceed to block (1905)).

At block (1903), if there are the data that induce cognitive compensation, the method could convert the data into output data that would allow compensatory cognitive process to take place. In one embodiment, the output data would allow the user's part of brain with preserved function could compensate for the ineffective part of brain's function. In one embodiment, the output data could be domain specific. In one embodiment, the system could identify and recognize faces, objects, texts, locations, positions from image data acquired. In one embodiment, the system could identify and recognize language information from the sound data acquired.

At block (1904), the method could output the converted data. In one embodiment, the methods can transmit data to the cognitive feedback module (107) to provide cognitive assistance to the user.

At block (1905), if there are the data that induce cognitive intervention, the method determines if the data meet the intervention condition(s). In one embodiment, the output data would allow the user's part of brain with preserved function could compensate for the ineffective part of brain's function. In one embodiment, the output data could be domain specific. In one embodiment, the system could identify and recognize faces, objects, texts, locations, positions from image data acquired. In one embodiment, the system could identify and recognize language information from the sound data acquired.

At block (1906), the method could execute user-specific cognitive intervention if intervention condition(s) are met. In one embodiment, the methods can transmit data to the cognitive feedback module (107) to execute the cognitive intervention.

In one embodiment, the cognitive assistance system (100) would have account management system that manage the users of the system, including medical professionals, care provides, family members, and/or patients.

In one embodiment the cognitive assistance system (100) could store the face data, voice data and/or other personal data of the users. In one embodiment, the system could use the face data, voice data and/or other personal data of the users to determine the person(s) utilizing the system. In one exemplary embodiment, the system could deny service to the person(s) utilizing the system if it determines that person is not authorized to use the system. In one exemplary embodiment, the system could provide individualized service toward a specific user. In one exemplary embodiment, the system could provide individualized service toward a specific user on a shared device.

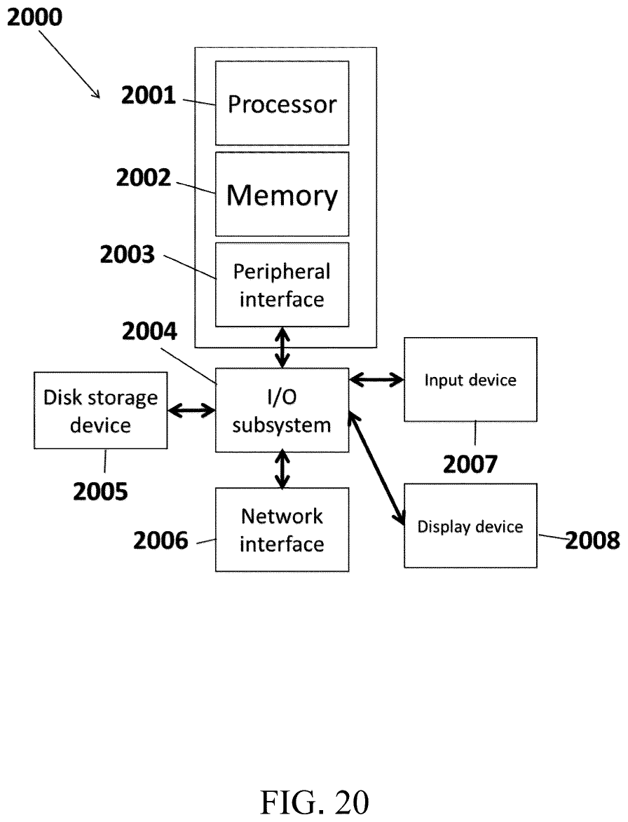

FIG. 20 and FIG. 1A depict a computer system (2000) that, in some embodiments, could serve as the system for the cognitive assistance system (100) to be operated on. The cognitive assistance system (100), in one embodiment, may be implemented in a computer system that includes one or more processors (2001), memory (2002), and a peripheral interface (2103). The memory (2002) may be of any type of medium capable of storing information accessible by the processor, is coupled to the processor, and storing instructions executable by the processor. The peripheral interface (1203) may be connected to input/output (I/O) subsystem (1204). The I/O subsystem may be connected to a disk storage device (2005), a network interface (2006), an input device (1207), a display device (1208), or other input/output devices. The input device (2007) can be a touch screen, or any other input devices. The above system is intended to represent a machine in the exemplary form of a computer system that, in some embodiments, capable of causing the machine to perform one or more methods discussed herein.

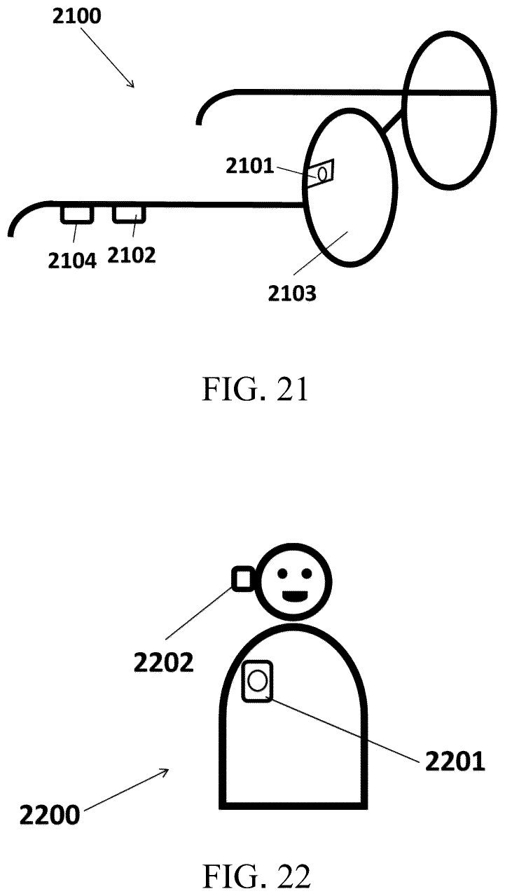

FIG. 21 and FIG. 1A depict a wearable cognitive prosthesis system (2100) that, in some embodiments, could serve as a cognitive assistance system (100). In one embodiment, the optical sensor (2101) can capture visual signals that can be used as input signals for the cognitive prosthesis system. In one embodiment, the microphone sensor (2202) can capture audio signal that can be used as input signal for the cognitive prosthesis system. In one embodiment, visual cognitive assistance information could be displayed on the display screen (2203). In one embodiment, audio cognitive assistance information could be provided through a speaker (2204).

FIG. 22 and FIG. 1A that provides customized cognitive assistance based on the patient's specific cognitive domain impairment depict a wearable cognitive prosthesis system (2200) that, in some embodiments, can serve as a cognitive assistance system (100). In one embodiment, the optical sensor (2201) can capture visual signals that can be used as input signals for the cognitive prosthesis system. In one embodiment, audio cognitive assistance information could be provided wirelessly through a speaker (2202).

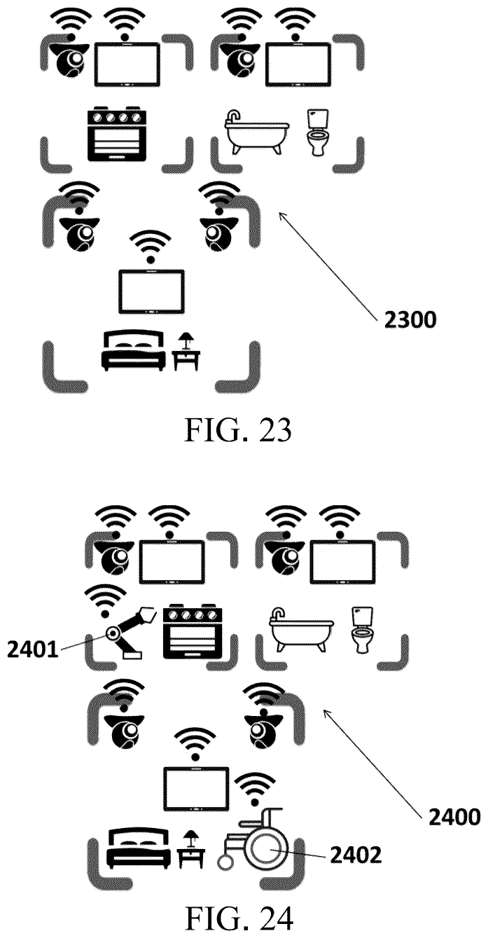

FIG. 23 and FIG. 1A depict an embodiment of the cognitive prosthesis system (2300) that, in some embodiments, can serve as a cognitive assistance system (100). In one embodiment, the devices could communicate through wired and/or wireless means.

FIG. 24 and FIG. 1A depict an embodiment of the cognitive prosthesis system (2400) that, in some embodiments, can serve as a cognitive assistance system (100). In one embodiment, the data input and/or data output could be through robotic (2401) and/or other electronic devices (2402).

In one embodiment, the cognitive assistance system (100) could communicate with hospital, authorities, family members.

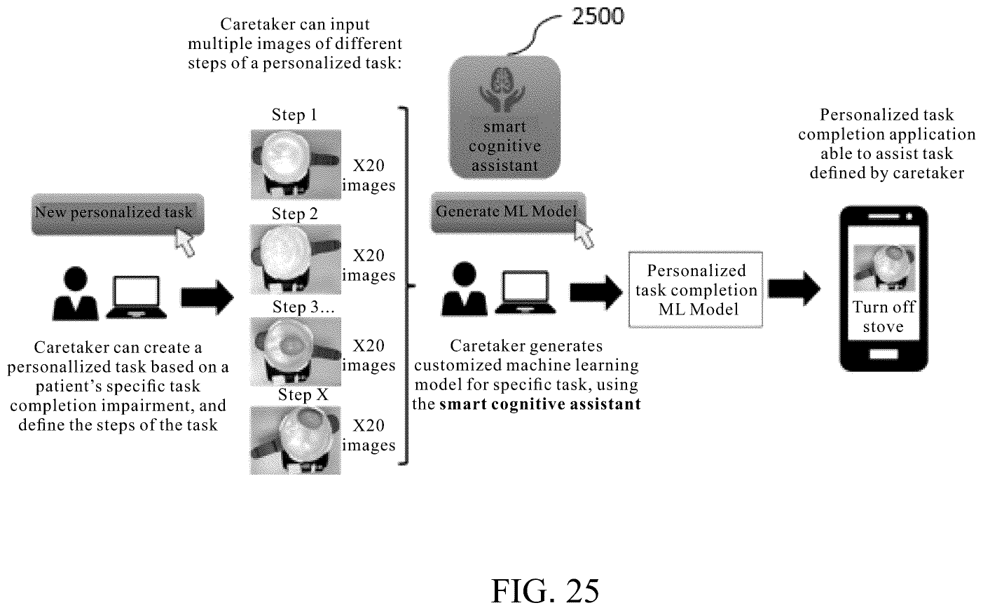

FIG. 25 illustrates an embodiment of a smart cognitive assistant (2500). A caretaker of a patient can use the smart cognitive assistant (2500) to create a personalized task module based on a patient's specific task completion impairment. A caretaker can define the tasks by inputting multiple images per a personalized task to train a machine learning module by the smart cognitive assistant (2500). For example, to create a task with four steps, the caretaker may input twenty images per step to the smart cognitive assistant (2500). The smart cognitive assistant (2500) would train a machine learning module using the eighty images for four steps. The trained personalized task module can be deployed into the system 1 illustrated in FIG. 1A as the data acquisition module (105), the data analysis module (106), and the cognitive feedback module (107).

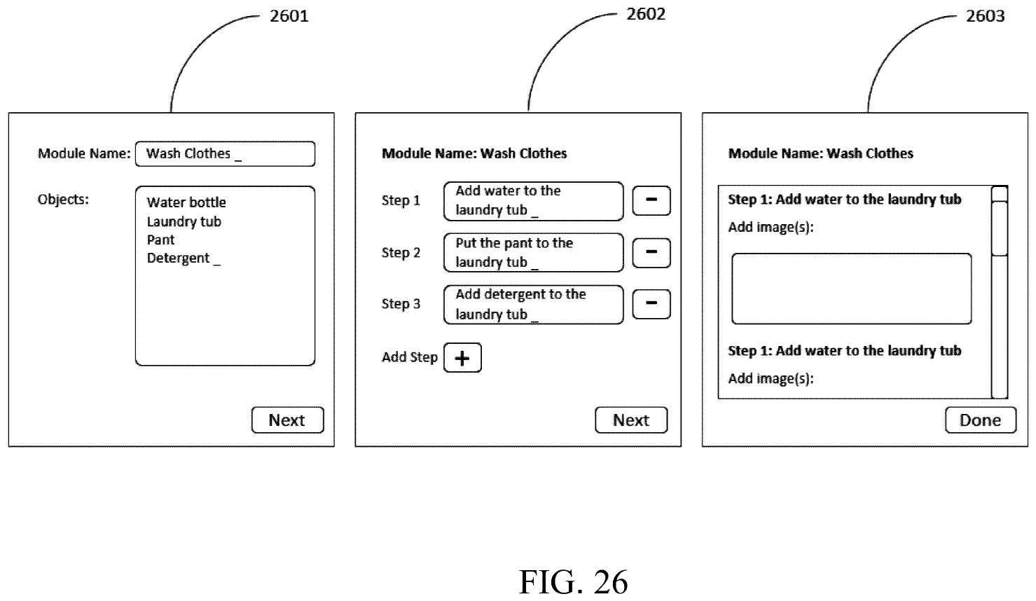

FIG. 26 shows exemplary output screens of the smart cognitive assistant (2500). To generate a personalized task module, a caretaker or a physician can input the name of the module and the objects used in the module using interface (2601). Then, the caretaker or the physician can input the descriptions of the steps of the module using interface (2602), and upload images for each step using interface (2603).

For each step of the module, the smart cognitive assistant (2500) may corelate the objects to be detected with the images uploaded, and optionally use the uploaded images to generate more images using a generative AI engine currently available. Then, the smart cognitive assistant (2500) may use the images uploaded and/or generated as a training dataset to train an AI module. The trained AI module may label the detected objects, and output the bounding boxes of the objects. With the coordinates of the bounding boxes, the positions of the objects in the images of each step of the module can be determined.

With the objects and their respective positions being determined, smart cognitive assistant (2500) can generate a basic set of new personalized task modules shown in FIG. 3. For example, the data acquisition module (105) would include AI detection modules that can detect the objects input by the caretaker or the physician using the interfaces (2601) to (2603). The data analysis module (106) would use the positions of the bounding boxes in the uploaded and/or generated images as the basis to determine if the user put a specific rehab tool at a specific position based on the image captured by the optical sensor (101) of the system (100). The texts and/or images input by the caretaker or the physician using the interfaces (2601) to (2603) can be used as the messages to be provide to the user by the cognitive feedback module (107).

With this basic set of new personalized task modules, the caretaker or the physician may set up more parameters. For example, the caretaker or the physician may set up the thresholds for different difficulty levels stored in the cognitive feedback module (107) shown in FIG. 5B, and change the contents of the texts and/or images, respectively.

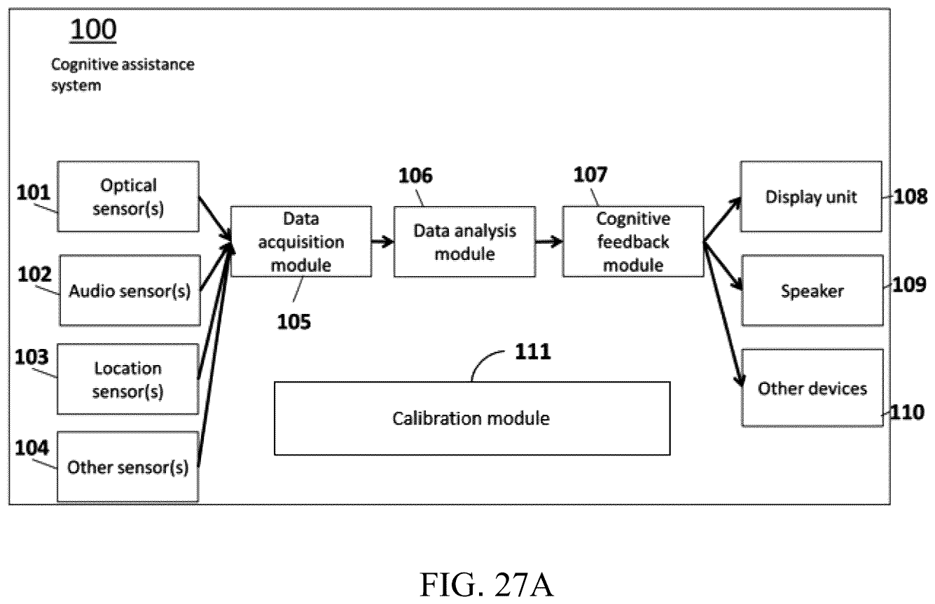

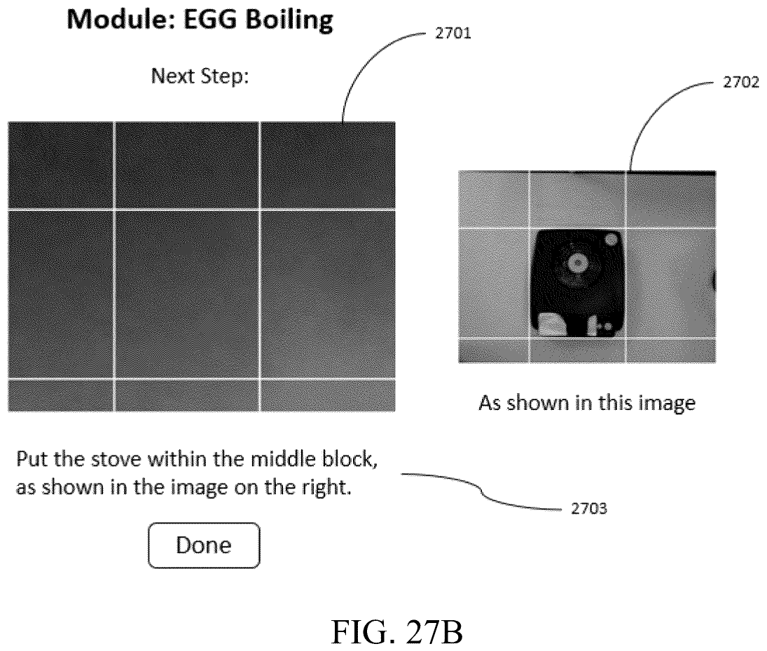

FIG. 27A illustrates the system (100) that further includes a calibration module (111), and FIG. 27B shows an exemplary screen output of the calibration module. The calibration module (111) may be used by the physician to calibrate the system (100) before the user use the system (100). Alternatively, the calibration module (111) may add additional steps for the user to operate during the rehab process.

For example, the calibration module (111) may ask the physician or the user to adjust the position of the camera to capture the whole surface with all rehab tools. The calibration module (111) may also ask the physician or the user to turn on the light of the camera, or to adjust brightness of the light to improve the object detection result. The calibration module (111) may further ask the physician or the user to put a specific rehab tool at a specific position to calibrate the data acquisition module (105) and/or the data analysis module (106) of the system (100). The calibration steps mentioned above may be performed by the physician as a setup procedure of the system (100) before the user use the system (100).

As shown in FIG. 27B, the system (100) may provide reference lines on the image (2701) shown to the user, provide a reference image (2702), and prompt a text message (2703) to the user, guiding him or her to put the stove within the middle block defined by the reference lines. When the user put the stove, the detection module of the data acquisition module (105) may start detecting whether stove is detected, and the data analysis module (106) may analyze to determine whether the stove is disposed within the correct position. Once the capture scope of the camera, brightness of the light and/or other environmental conditions are set up correctly, the calibration module (111) would determine that the system (100) is well calibrated, and therefore is ready to use.

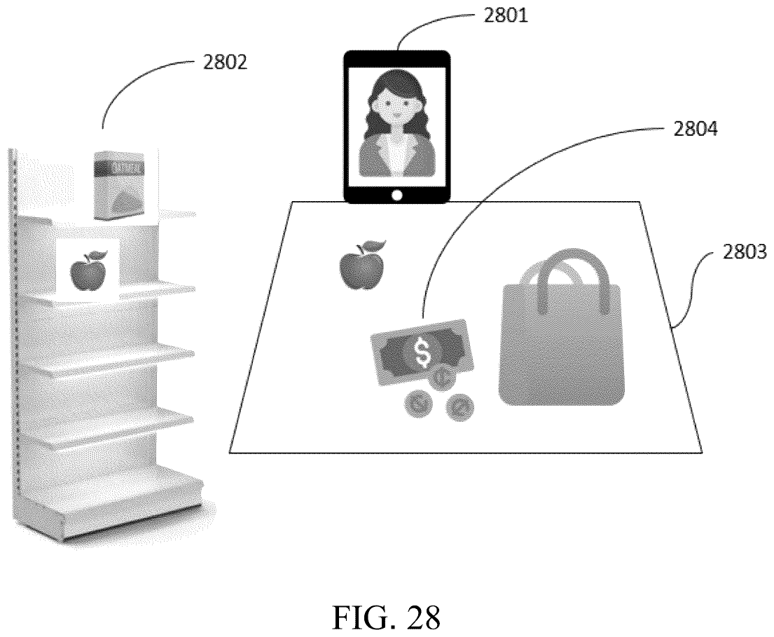

FIG. 28 and FIG. 1A show an example of a “shopping” module including a set of rehab tools, which module allows a user to perform more complicated rehab tasks. In this example, the system (100) is implemented in a tablet (2801) with a camera and a touch-sensitive display. The rehab tools (2804), including different groceries, a shopping bag and money, may be placed on a shelf (2082) or a table surface (2803). The camera of the tablet (2801) can capture the images of the whole shopping premise, including the shelf (2082), the table surface (2803) and all rehab tools (2804).

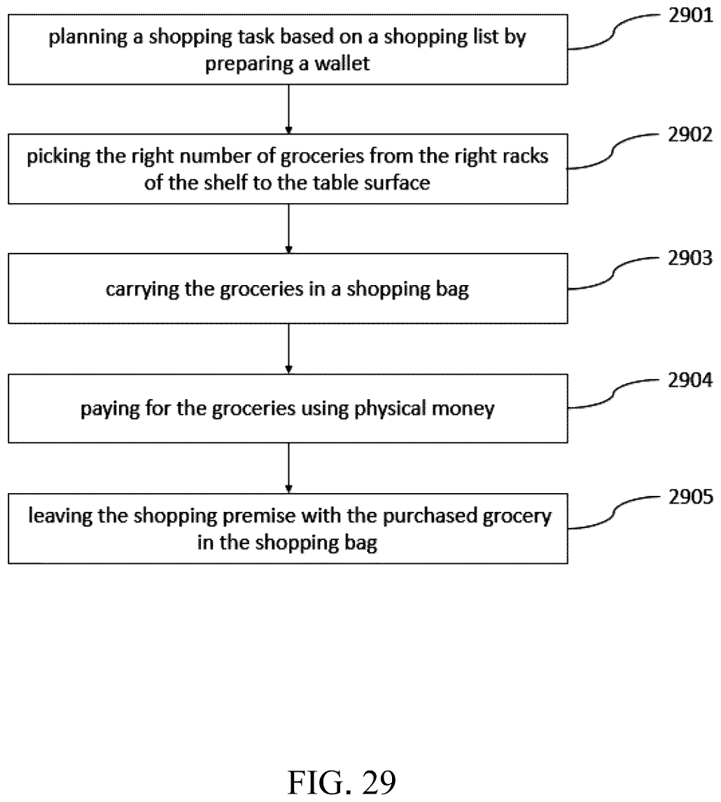

FIG. 29, FIG. 28 and FIG. 1A show the steps of the process of this shopping module. The tablet (2801) may instruct the user to perform complex steps, including: planning a shopping task based on a shopping list by preparing a wallet (2901), picking the right number of groceries from the right racks of the shelf to the table surface (2902), carrying them in a shopping bag (2903), paying for the groceries using physical money (2904), and/or leaving the shopping premise with the purchased grocery in the shopping bag (2905). The system (100) implemented by the tablet (2801) may instruct the user to perform those steps by texts, images and/or audio messages, and may use AI visions to detect the gesture or the voice of the user.

Different from the simple task module such as boiling eggs, this shopping module may evaluate more complex executive functions of the user, including “gestures” and/or “actions” of the user, and even “simple dialogue” between the system (100) and the user. Such complex task module trains different executive functions of the user more effectively, and evaluate the cognitive states of the user more accurately.

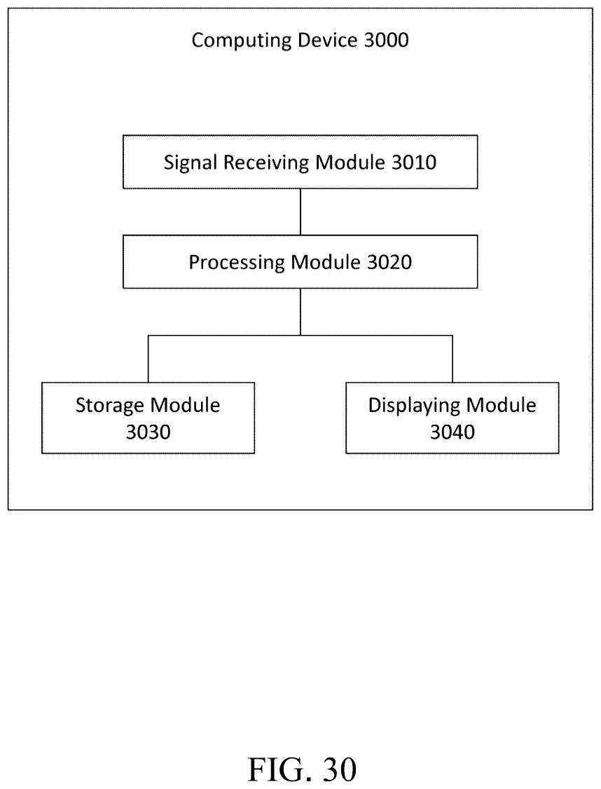

FIG. 30 illustrates a computing device (3000) for providing cognitive training. The computing device (3000) could include a signal receiving module (3010), a processing module (3020), a storage module (3030), and a displaying module (3040).

The signal receiving module (3010) could be configured to receive the input data. The input data could be, for example, a feedback response, but is not limited thereto. In some embodiments, the computing device (3000) could receive the feedback response by a hey press. In some embodiments, the computing device (3000) could use a gyroscope in order to detect and maintain the direction. That is, the computing device (3000) can detect and maintain the direction corresponding to a received image by using the gyroscope.

The processing module (3020) could be configured to couple with the signal receiving module (3010) and could be configured to execute the code stored in the storage module (3030) such that the processing module (3020) is able to carry out the method for providing cognitive training. The processing module (3020) could be a finished product known to a person having ordinary knowledge in the art, which may be specifically composed of one or more central processing units, but is not limited thereto.

The storage module (3030) could be configured to couple with the processing module (3020) and be configured to store the code to be executed by the processing module (3020). The storage module (3030) could be a finished product known to a person having ordinary knowledge in the art, which could be specifically composed of volatile memory and non-volatile memory, but is not limited thereto. The volatile memory could be a finished product known to a person having ordinary knowledge in the art, such as dynamic random access memory or static random access memory, but is not limited thereto. The non-volatile memory could be a finished product known to persons having ordinary knowledge in the art, such as read-only memory, flash memory or non-volatile random access memory, but is not limited thereto.

The displaying module (3040) could be configured to couple with the processing module (3020) and be configured to display a first target object, a second target object, and/or another target object. The displaying module (3040) could be could be a finished product known to a person having ordinary knowledge in the art, such as a displayer, but is not limited thereto.

The computing device (3000) for providing cognitive training could be configured to carry out any one of the methods for providing cognitive training by executing the code stored in the storage module (3030) by the processing module (3020). Thereby, the computing device (3000) can provide the cognitive training for many different users who need, and then it makes the cognitive training be more efficient. Besides, the computing device (3000) can provide a suitable cognitive training based on the user who needs, and then it makes the cognitive training be more effective.

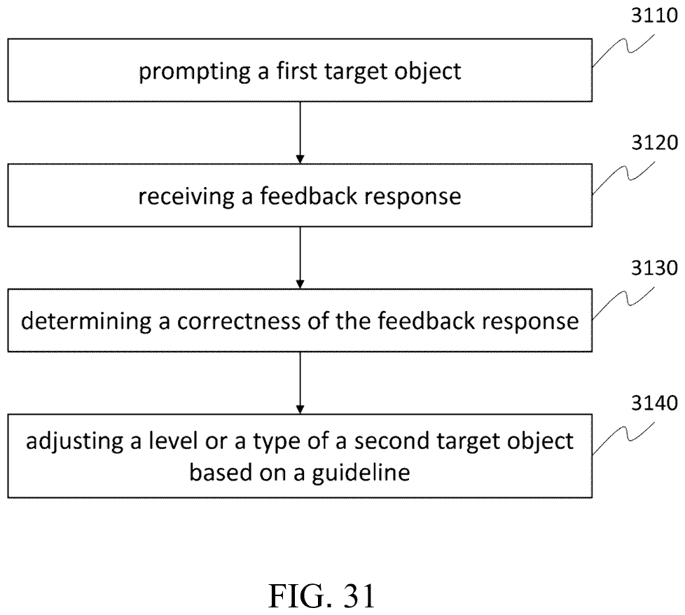

FIG. 31 illustrates a method for providing cognitive training. The method as shown in FIG. 31 could include blocks (3110), (3120), (3130), and (3140). In some embodiments, the method could be carrier out by using the computing device (3000) as shown in FIG. 30.

At the block (3110), prompt a first target object for recognizing by a user. In some embodiments, a computing device could prompt the first target object for recognizing by the user. In some embodiments, the first target object could comprise at least one of a target image, a target text, and a target voice. In some embodiments, the first target object could be determined based on the user.

In some embodiments, the target image could be a facial image for person identification, relationship identification (with user or others), or face-features classification ability (male/female, race, or age), etc. In some embodiments, the target text could be a character string for understanding the meanings by context and/or inference (adding the elements that are used for testing reading comprehension). In some embodiments, the target voice could be a voice contents, such as a voice content from a person with relationship or a voice content with features (old, gender, accent, speed).

In some embodiments, the first target object could be determined by the computing device. In some embodiments, the first target object could be determined based on at least one of a magnetic resonance imaging (MRI) of the user, medical records of the user, and a result of the shopping module which has been performed by the user (or any other rehab result of the rehab module which has been performed by the user).

At the block (3120), receive a feedback response from the user by the computing device. That is, a computing device could receive a feedback response from the user. In some embodiments, the feedback response could comprise at least one of an image response, a text response, and a voice response.

At the block (3130), determine a correctness of the feedback response by comparing the feedback response with a stored answer by the computing device. That is, a computing device could compare the feedback response with a stored answer in order to determine a correctness of the feedback response. In some embodiments, the stored answer could comprise at least one of a primary answer and a secondary answer. The primary answer could refer to a correct answer, such as the correct name corresponding to the target image. The secondary answer could refer to a relationship to the correct answer. In an example, the primary answer is the name (such as John) and the secondary answer is the relationship (such as son) when the first target object is a target image for person identification.

In some embodiments, the correctness of the feedback response could comprise at least one of a match result that the feedback response exactly matches with the stored answer or not and a match ratio that the feedback response is close to the stored answer.

At the block (3140), adjust a level or a type of a second target object based on a guideline associated with the correctness of the feedback response. In some embodiments, the guideline is that the level of the second target object is upgraded when the match result is that the feedback response exactly matches with the stored answer or when the match ratio is greater than or equal to a threshold, and the level of the second target object is downgraded when the match result is that the feedback response does not exactly match with the stored answer or when the match ratio is less than the threshold. In some embodiments, the guideline is that the type of the second target object which is more difficult than the first target object is determined when the match result is that the feedback response exactly matches with the stored answer or when the match ratio is greater than or equal to a threshold, and the type of the second target object which is easier than the first target object is determined when the match result is that the feedback response does not exactly match with the stored answer or when the match ratio is less than the threshold.

Thereby, the method for providing cognitive training can not only efficiently provide the cognitive training for the user but also can adjust the next cognitive training for the user based on the guideline and the correctness of the feedback response. That is, the method can provide a suitable cognitive training for the user based on the guideline and the correctness of the feedback response by the computing device.

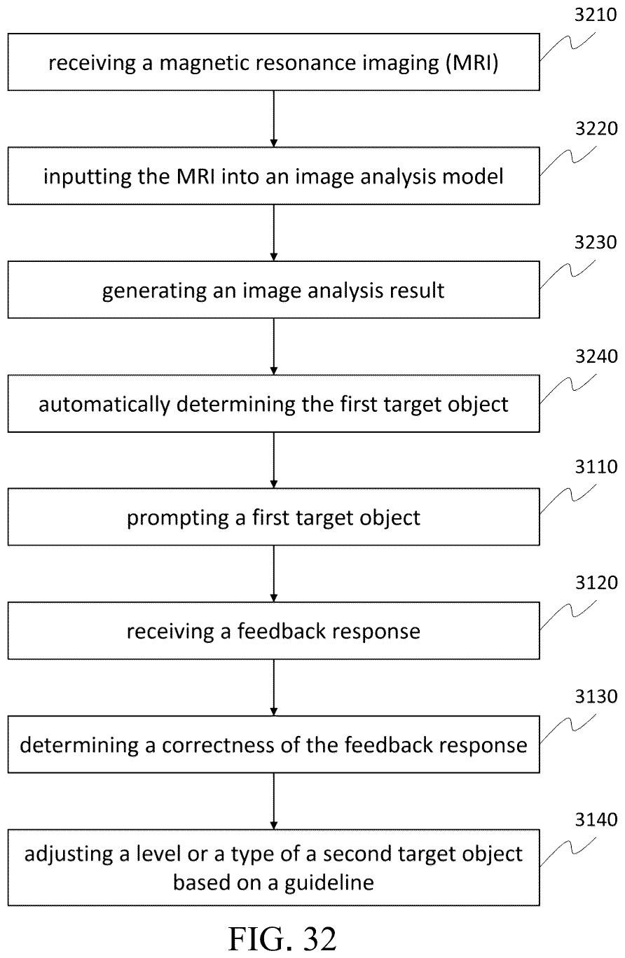

FIG. 32 illustrates another method for providing cognitive training. The method as shown in FIG. 32 could include blocks (3110), (3120), (3130), (3140), (3210), (3220), (3230) and (3240), wherein the blocks (3110), (3120), (3130), and (3140) are substantially the same as those shown in FIG. 31. That is, the method as shown in FIG. 32 may include the blocks (3110), (3120), (3130), and (3140), which are substantially the same as those shown in FIG. 31, and further include the blocks (3210), (3220), (3230) and (3240).

At the block (3210), receive a magnetic resonance imaging (MRI) of the user. In some embodiments, the method could also receive medical records of the user. That is, the block (3210) could refer to receive an original data, such as the MRI of the user or the medical records of the user.

At the block (3220), input the MRI into an image analysis model. In some embodiments, the image analysis model could be a trained artificial intelligent model which has been trained by plural pieces of data such that the image analysis model is able to analyze the inputted image. Each of plural pieces of data could comprise a training image and a training information corresponding to the training image, especially an MRI and an information corresponding to the MRI.

At the block (3230), an image analysis result corresponding to the MRI is generated in real time by the image analysis model. That is, the image analysis result corresponding to the MRI could be automatically generated by the image analysis model after the MRI is inputted into the image analysis model.

At the block (3240), automatically determine the first target object based on the image analysis result by the computing device. That is, the first target object for recognizing by a user could be determined based on the image analysis result by the computing device.

Thereby, the method for providing cognitive training can not only efficiently provide the cognitive training for the user but also can initially provide a suitable cognitive training for the user based on the image analysis result by the computing device. Besides, the method can also provide a suitable cognitive training for the user based on the guideline and the correctness of the feedback response by the computing device after the current cognitive training has been finished.

The computing device disclosed herein uses the feedback from the user to optimized the treatment process, for example, for providing a refined and more precise questions for further determine the status of the user or progress of the user, which in term is used to refine the formula or treatment model in the computing device.

In some embodiments, the steps of the method for providing cognitive training as described above may be stored in a non-transitory computer-readable recording medium in a series of particular codes or a series of particular instruction sets, the non-transitory computer-readable recording medium may be, for example, a hard disk, a CD-ROM, a magnetic disk, or a USB disk, but is not limited thereto. After a computing device loading and executing the codes or the instruction sets stored in the non-transitory computer-readable recording medium, the non-transitory computer-readable recording medium is able to complete steps such that any one of the methods for providing cognitive training as described above is carried out.

While the present disclosure has been described by means of specific embodiments, numerous modifications and variations could be made thereto by those skilled in the art without departing from the scope and spirit of the present disclosure set forth in the claims. Therefore, the protection of this application shall be as defined in the claims instead of the contents disclosed in the specification.

What is claimed is:

1. A method for providing cognitive training by using a computing device, comprising:
prompting a first target object for recognizing by a user;
receiving a feedback response from the user by the computing device;
determining a correctness of the feedback response by comparing the feedback response with a stored answer by the computing device; and
adjusting a level or a type of a second target object based on a guideline associated with the correctness of the feedback response,
wherein the second target object is provided for recognizing by the user after the first target object is provided.

2. The method according to claim 1, wherein the first target object comprises at least one of a target image, a target text, and a target voice.

3. The method according to claim 1, wherein the correctness of the feedback response comprises at least one of a match result that the feedback response exactly matches with the stored answer or not and a match ratio that the feedback response is close to the stored answer.

4. The method according to claim 3, wherein the guideline is that
the level of the second target object is upgraded when the match result is that the feedback response exactly matches with the stored answer or when the match ratio is greater than or equal to a threshold, and
the level of the second target object is downgraded when the match result is that the feedback response does not exactly match with the stored answer or when the match ratio is less than the threshold.

5. The method according to claim 3, wherein the guideline is that
the type of the second target object which is more difficult than the first target object is determined when the match result is that the feedback response exactly matches with the stored answer or when the match ratio is greater than or equal to a threshold, and
the type of the second target object which is easier than the first target object is determined when the match result is that the feedback response does not exactly match with the stored answer or when the match ratio is less than the threshold.

6. The method according to claim 1, wherein the stored answer comprises at least one of a primary answer and a secondary answer.

7. The method according to claim 1, wherein the prompting the first target object for recognizing by the user is performed by the computing device.

8. The method according to claim 1, further comprising:
receiving a magnetic resonance imaging (MRI) of the user;
inputting the magnetic resonance imaging into an image analysis model, and then an image analysis result corresponding to the magnetic resonance imaging being generated in real time by the image analysis model; and
automatically determining the first target object based on the image analysis result by the computing device.

9. A computing device for providing cognitive training, comprising:
a signal receiving module;
a processing module, configured to couple with the signal receiving module;
a storage module, configured to couple with the processing module; and
a displaying module, configured to couple with the processing module,
wherein a code being stored in the storage module, and after the processing module executing the code stored in the storage module, the computing device being able to execute steps as described below:
prompting a first target object for recognizing by a user;
receiving a feedback response from the user by the computing device;
determining a correctness of the feedback response by comparing the feedback response with a stored answer by the computing device; and
adjusting a level or a type of a second target object based on a guideline associated with the correctness of the feedback response,
wherein the second target object is provided for recognizing by the user after the first target object is provided.

10. The computing device according to claim 9, wherein the first target object comprises at least one of a target image, a target text, and a target voice.

11. The computing device according to claim 9, wherein the correctness of the feedback response comprises at least one of a match result that the feedback response exactly matches with the stored answer or not and a match ratio that the feedback response is close to the stored answer.

12. The computing device according to claim 11, wherein the guideline is that
the level of the second target object is upgraded when the match result is that the feedback response exactly matches with the stored answer or when the match ratio is greater than or equal to a threshold, and
the level of the second target object is downgraded when the match result is that the feedback response does not exactly match with the stored answer or when the match ratio is less than the threshold.

13. The computing device according to claim 11, wherein the guideline is that
the type of the second target object which is more difficult than the first target object is determined when the match result is that the feedback response exactly matches with the stored answer or when the match ratio is greater than or equal to a threshold, and
the type of the second target object which is easier than the first target object is determined when the match result is that the feedback response does not exactly match with the stored answer or when the match ratio is less than the threshold.

14. The computing device according to claim 9, wherein the stored answer comprises at least one of a primary answer and a secondary answer.

15. The computing device according to claim 9, wherein the prompting the first target object for recognizing by the user is performed by the computing device.

16. The computing device according to claim 9, further comprising:
receiving a magnetic resonance imaging (MRI) of the user;
inputting the magnetic resonance imaging into an image analysis model, and then an image analysis result corresponding to the magnetic resonance imaging being generated in real time by the image analysis model; and
automatically determining the first target object based on the image analysis result by the computing device.

17. A non-transitory computer-readable recording medium capable of providing cognitive training, after a computing device loading and executing a code stored in the non-transitory computer-readable recording medium, the non-transitory computer-readable recording medium being able to complete steps as described below:
prompting a first target object for recognizing by a user;
receiving a feedback response from the user by the computing device;
determining a correctness of the feedback response by comparing the feedback response with a stored answer by the computing device; and
adjusting a level or a type of a second target object based on a guideline associated with the correctness of the feedback response,
wherein the second target object is provided for recognizing by the user after the first target object is provided.

18. The non-transitory computer-readable recording medium according to claim 17, wherein the first target object comprises at least one of a target image, a target text, and a target voice.

19. The non-transitory computer-readable recording medium according to claim 17, wherein the correctness of the feedback response comprises at least one of a match result that the feedback response exactly matches with the stored answer or not and a match ratio that the feedback response is close to the stored answer.

20. The non-transitory computer-readable recording medium according to claim 19, wherein the guideline is that
the level of the second target object is upgraded when the match result is that the feedback response exactly matches with the stored answer or when the match ratio is greater than or equal to a threshold, and
the level of the second target object is downgraded when the match result is that the feedback response does not exactly match with the stored answer or when the match ratio is less than the threshold.

21. The non-transitory computer-readable recording medium according to claim 19, wherein the guideline is that
the type of the second target object which is more difficult than the first target object is determined when the match result is that the feedback response exactly matches with the stored answer or when the match ratio is greater than or equal to a threshold, and
the type of the second target object which is easier than the first target object is determined when the match result is that the feedback response does not exactly match with the stored answer or when the match ratio is less than the threshold.

22. The non-transitory computer-readable recording medium according to claim 17, wherein the stored answer comprises at least one of a primary answer and a secondary answer.

23. The non-transitory computer-readable recording medium according to claim 17, wherein the prompting the first target object for recognizing by the user is performed by the computing device.

24. The non-transitory computer-readable recording medium according to claim 17, further comprising:
receiving a magnetic resonance imaging (MRI) of the user;
inputting the magnetic resonance imaging into an image analysis model, and then an image analysis result corresponding to the magnetic resonance imaging being generated in real time by the image analysis model; and
automatically determining the first target object based on the image analysis result by the computing device.
